(19) United States
(12) Patent Application Publication
(10) Pub. No.: US 2025/0311913 A1
(43) Pub. Date: Oct. 9, 2025

(54) SIDE-VIEWING ENDOSCOPE CAP

(71) Applicant: EndoTheia, Inc., Nashville, TN (US)

(72) Inventors: Joshua Gafford, Nashville, TN (US); Patrick Anderson, Nashville, TN (US); Scott Webster, Nashville, TN (US); Mina Schaafsma, Nashville, TN (US); Tyler Efird, Nashville, TN (US); Jacob Bumpus, Gallatin, TN (US)

(21) Appl. No.: 19/169,823

(22) Filed: Apr. 3, 2025

Related U.S. Application Data
(60) Provisional application No. 63/574,673, filed on Apr. 4, 2024.

(51) Int. Cl. A61B 1/00; A61B 1/06

(52) U.S. Cl. CPC A61B 1/00137; A61B 1/00098; A61B 1/00133; A61B 1/0623

(57) ABSTRACT

The present disclosure provides for an endoscope apparatus. The apparatus may include endcap having a body and a side opening. The body may be configured to fit onto a distal end of an endoscope. The endoscope may include a forward-facing working port and a tool that is configured to be advanced out of the working port. In some embodiments, the apparatus includes an image sensor disposed on the body of the endcap. In some embodiments, the apparatus includes an elevator pivotably coupled to the body of the endcap. The elevator may be configured to be pivoted relative to the body such that the elevator deflects the tool toward the side opening of the endcap into the lateral workspace. In some embodiments, the apparatus further includes a user interface and a pull-wire. The pull-wire may be coupled to the user interface and the elevator.

### CROSS-REFERENCES TO RELATED APPLICATIONS

This application is a non-provisional of and claims priority to U.S. Provisional Application No. 63/574,673, filed Apr. 4, 2024, entitled “SIDE-VIEWING ENDOSCOPE CAP,” which is hereby incorporated by reference in its entirety.

### STATEMENT REGARDING FEDERALLY SPONSORED RESEARCH OR DEVELOPMENT

Not Applicable.

### REFERENCE TO SEQUENCE LISTING OR COMPUTER PROGRAM LISTING APPENDIX

Not Applicable.

### BACKGROUND

The present disclosure relates to endoscopic surgical procedures. More particularly, the present disclosure relates to apparatuses and methods of enhancing endoscope devices for performing Endoscopic Retrograde Cholangeopancreatography or other procedures.

Endoscopic Retrograde Cholangeopancreatography (ERCP) is a procedure where the bile ducts are accessed from the duodenum using a flexible endoscope, and is the gold standard treatment for a variety of disorders of the pancreaticobiliary system. There are 700,000 ERCPs performed annually in the United States alone. In ERCP, a specialized flexible endoscope (called a duodenoscope) enters the body trans-orally and navigates down into the stomach, through the pylorus and into the duodenum. Then, various flexible tools are passed through the duodenoscope and aimed sideways at its tip, under side view camera visualization, using the elevator mechanism built into the duodenoscope tip. The elevator is a pullwire-actuated lever residing in the tip of the duodenoscope, at the outlet of the working channel, and is used to deflect various tools (guidewires, stents, balloons, sphincterotomes) laterally to facilitate cannulation of the major or minor duodenal ampulla, which is where the bile duct empties into the duodenum.

An FDA review in 2019 uncovered numerous post-ERCP deaths and infections due to multi-drug-resistant bacteria transferred between patients undergoing ERCP. The cause of the infections was determined to be the duodenoscopes, which were transmitting bacteria including Escherichia coli, Staphylococcus, and carbapenem-resistant Enterobac-teriacae between patients in 5.4% of all cases. Furthermore, the FDA linked the high infection rates directly to the elevator mechanism. This elevator mechanism consists of several tiny, moving parts with small gaps that harbor dangerous bacteria and are difficult to clean and sterilize between cases. These infections result not only from failure to follow manufacturer cleaning guidelines; a United States Senate study found that contamination can happen even when all cleaning and sterilization processing steps are correctly followed, meaning that prevention of infection is extremely challenging.

It would be advantageous to provide an endoscope, or components thereof, that provides suitable functionality for ERCP purposes, while addressing the aforementioned issues regarding infections. What is needed, then, are improvements in apparatuses and methods for endoscopes.

### BRIEF SUMMARY

This Brief Summary is provided to introduce a selection of concepts in a simplified form that are further described below in the Detailed Description. This Summary is not intended to identify key features or essential features of the claimed subject matter, nor is it intended to be used as an aid in determining the scope of the claimed subject matter.

The present disclosure provides for an endoscope apparatus. The apparatus may include endcap having a body and a side opening. The body may be configured to fit onto a distal end of an endoscope. The endoscope may include a forward-facing working port and a tool that is configured to be advanced out of the working port.

In some embodiments, the apparatus includes an image sensor disposed on the body of the endcap. The image sensor may be configured to visualize a workspace that is lateral of both the endcap and the distal end of the endoscope. The image sensor may be positioned on an interior of the body of the endcap, such that the image sensor visualizes the lateral workspace through the side opening of the endcap. In some embodiments, the image sensor includes a complimentary metal-oxide semiconductor array. The apparatus may further include a light source disposed on the body of the endcap and configured to illuminate the lateral workspace.

In some embodiments, the apparatus includes an elevator pivotably coupled to the body of the endcap. The elevator may be configured to be pivoted relative to the body such that the elevator deflects the tool toward the side opening of the endcap into the lateral workspace. The elevator may be configured to deflect the tool until the tool is secured in place between the elevator and an edge of the side opening.

In some embodiments, the apparatus further includes a user interface and a pull-wire. The pull-wire may be coupled to the user interface and the elevator. The user interface may be configured to displace the pull-wire. The elevator may be pivoted relative to the body of the endcap in response to displacement of the pull-wire. In some embodiments, the elevator is configured to be pivoted towards the working port of the endoscope when the pull-wire is displaced towards the user interface, and the elevator is configured to be pivoted away from the working port of the endoscope when the pull-wire is displaced away from the user interface. In some embodiments, the user interface is secured to the endoscope.

Another aspect of the present disclosure is a method of performing surgery. The method may include providing an endcap having a body and a side opening. The method may include securing the body of the endcap onto a distal end of an endoscope. The endoscope may include a forward-facing camera and working port. The method may further include advancing an endoscopic tool out of the working port. The method may include visualizing, via an image sensor disposed on the body of the endcap, a workspace that is lateral of both the endcap and the distal end of the endoscope. The method may include pivoting an elevator coupled to the body of the endcap, such that the elevator deflects the tool towards the side opening of the endcap and into the lateral workspace.

In some embodiments, the method includes securing a user interface to the endoscope and providing a pull-wire coupled to the user interface and the elevator. The method may further include displacing the pull-wire via the user interface, such that the elevator is pivoted relative to the body of the endcap in response to displacement of the pull-wire. The elevator may be configured to be pivoted relative to the body of the endcap in response to displacement of the pull-wire.

Numerous other objects, advantages and features of the present disclosure will be readily apparent to those of skill in the art upon a review of the following drawings and description of a preferred embodiment.

### BRIEF DESCRIPTION OF THE DRAWINGS

FIG. 1 is a schematic illustration of a patient's biliary system, according to some embodiments of the present disclosure.

FIG. 2 is an endoscope apparatus configured to facilitate implementation and use of a removable endcap on a traditional endoscope, including the endcap and a user interface, according to some embodiments of the present disclosure.

FIG. 3 is a video processing unit for the apparatus of FIG. 2, according to some embodiments of the present disclosure.

FIG. 4 is a perspective view of the apparatus of FIG. 2, implemented on a traditional endoscope, according to some embodiments of the present disclosure.

FIG. 5 is a detailed perspective view of the endcap of FIG. 2 with an elevator thereof in an engaged orientation, according to some embodiments of the present disclosure.

FIG. 6 is a detailed perspective view of the endcap of FIG. 2 being fitted onto an endoscope, according to some embodiments of the present disclosure.

FIG. 7 is a detailed perspective view of the endcap of FIG. 2 with an elevator thereof in an unengaged orientation, according to some embodiments of the present disclosure.

FIG. 8 is a detailed perspective view of the endcap of FIG. 2 with an endoscope tool extended therethrough, according to some embodiments of the present disclosure.

FIG. 9 is a detailed perspective view of the endcap of FIG. 2 with an elevator thereof shown being pivoted relative to the endcap, according to some embodiments of the present disclosure.

FIG. 10 is a side cross-sectional view of the endcap of FIG. 2, according to some embodiments of the present disclosure.

FIG. 11 is a side view of the endcap of FIG. 2 with flexural clamping elements for fitting the endcap on an endoscope, according to some embodiments of the present disclosure.

FIG. 12 is a cross-sectional view of the endcap of FIG. 11, according to some embodiments of the present disclosure.

FIG. 13 is a side view of the endcap of FIG. 2 with an over-molded press-fit interface for fitting the endcap on an endoscope, according to some embodiments of the present disclosure.

FIG. 14 is a cross-sectional view of the endcap of FIG. 13, according to some embodiments of the present disclosure.

FIG. 15 is a detailed perspective view of the user interface of FIG. 2, according to some embodiments of the present disclosure.

FIG. 16 is an exploded view of the user interface of FIG. 2, according to some embodiments of the present disclosure.

FIG. 17 is a detailed view of the user interface of FIG. 2 with a knob being operated, according to some embodiments of the present disclosure.

FIG. 18 is a detailed perspective view of the endcap of FIG. 2 with an elevator thereof being pivoted towards an unengaged orientation, according to some embodiments of the present disclosure.

FIG. 19 is a detailed view of the user interface of FIG. 2 with a knob being operated, according to further embodiments of the present disclosure.

FIG. 20 is a detailed perspective view of the endcap of FIG. 2 with a elevator thereof being pivoted towards a fully engaged orientation, according to some embodiments of the present disclosure.

FIG. 21 is a side cross-sectional view of the user interface of FIG. 2, according to some embodiments of the present disclosure.

FIG. 22 is a side cross-sectional view of the user interface of FIG. 2, according to further embodiments of the present disclosure.

FIG. 23 is a side cross-sectional view of the user interface of FIG. 2, according to further embodiments of the present disclosure.

FIG. 24 is a side cross-sectional view of the user interface of FIG. 2, according to further embodiments of the present disclosure.

FIG. 25 is a side cross-sectional view of the user interface of FIG. 2, according to further embodiments of the present disclosure.

FIG. 26 is a detailed perspective view of the user interface of FIG. 2, according to further embodiments of the present disclosure.

FIG. 27 is an exploded view of the user interface of FIG. 2, according to further embodiments of the present disclosure.

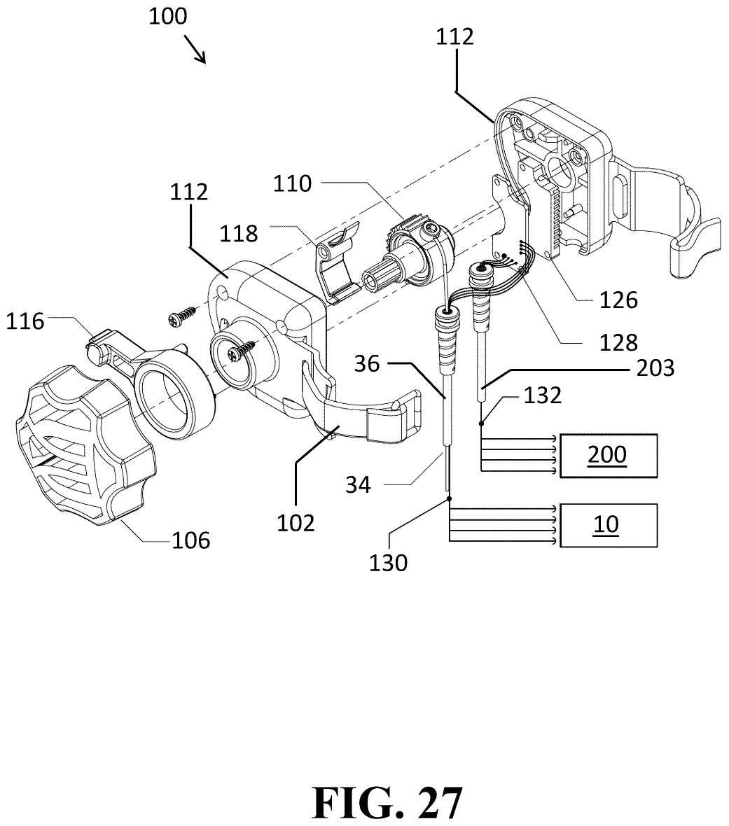

FIG. 28 is a detailed perspective view of the endcap of FIG. 2, according to other embodiments of the present disclosure.

FIG. 29 is a detailed perspective view of the endcap of FIG. 2 being fitted onto an endoscope via an intermediate sleeve, according to some embodiments of the present disclosure.

FIG. 30 is a detailed perspective view of the endcap of FIG. 2 being fitted onto an endoscope via a clasp, according to some embodiments of the present disclosure.

FIG. 31 is a detailed perspective view of the endcap of FIG. 30 with the clasp in an open arrangement, according to some embodiments of the present disclosure.

FIG. 32 is a detailed perspective view of the endcap of FIG. 2 being fitted onto an endoscope via a clasp with a living hinge, according to some embodiments of the present disclosure.

FIG. 33 is a perspective view of the apparatus of FIG. 2, implemented on a traditional endoscope, according to other embodiments of the present disclosure.

FIG. 34 is a detailed view of the user interface of FIG. 2 with a signal mechanism, according to some embodiments of the present disclosure.

### DETAILED DESCRIPTION

While the making and using of various embodiments of the present invention are discussed in detail below, it should be appreciated that the present invention provides many applicable inventive concepts that are embodied in a wide variety of specific contexts. The specific embodiments discussed herein are merely illustrative of specific ways to make and use the invention and do not delimit the scope of the invention. Those of ordinary skill in the art will recognize numerous equivalents to the specific apparatus and methods described herein. Such equivalents are considered to be within the scope of this invention and are covered by the claims.

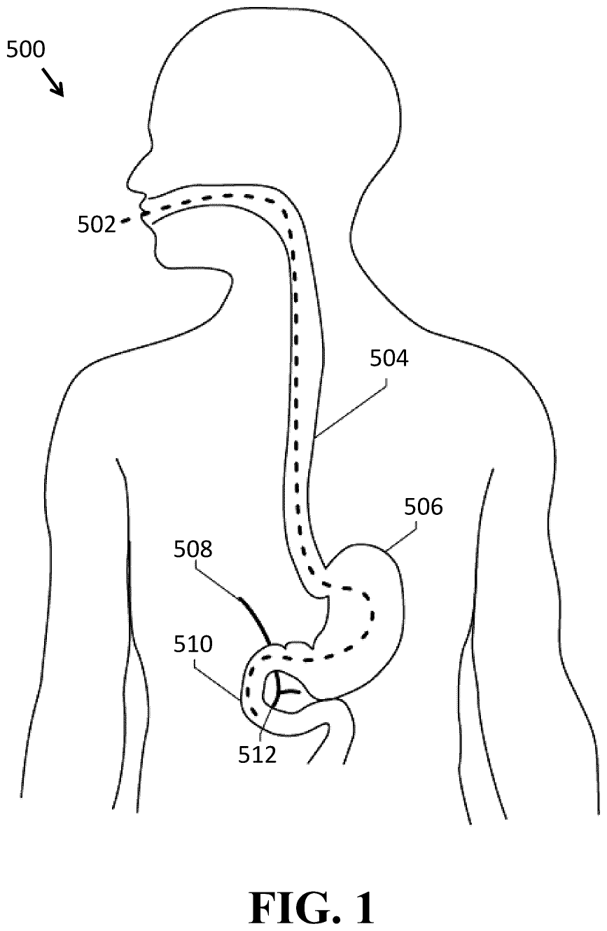

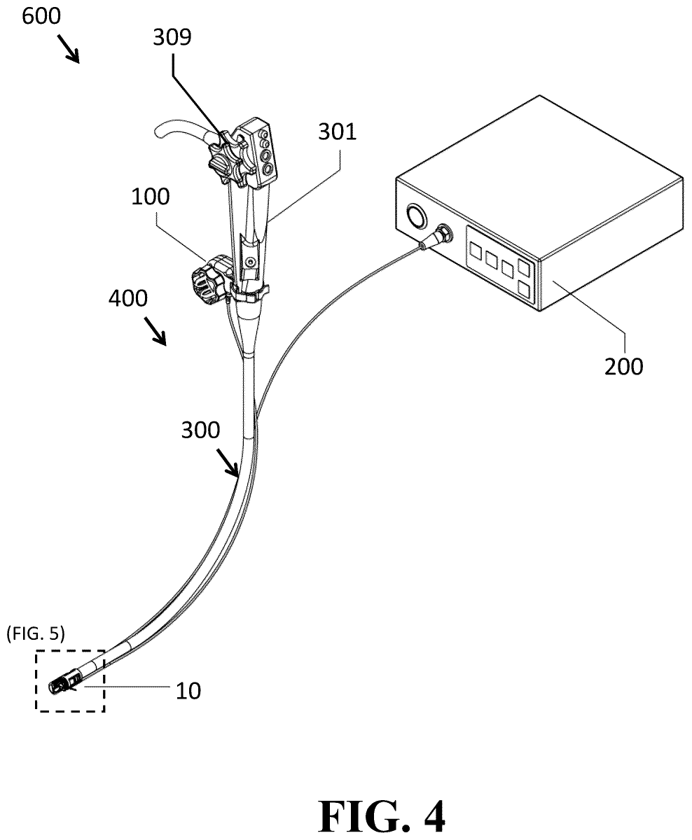

The present disclosure provides apparatuses and methods for performing surgical procedures, including ERCP, using standard, reusable or disposable forward-viewing endoscopes—such as an endoscope 300 as shown with reference to FIG. 4 (e.g., a duodenoscope, a gastroscope, a colonoscope, etc.)—which are easier to clean and sterilize when compared to reusable duodenoscopes used in conventional ERCP procedures. Referring now to FIG. 1, a patient's biliary apparatus 500 is shown, according to some embodiments of the present disclosure. As suggested above, in ERCP, an endoscope enters the body trans-orally via an endoscope path 502 and navigates down through an esophagus 504 into a stomach 506, thereby providing access to a biliary tract 508, a duodenum 510, and a duodenal ampulla 512. The apparatuses and methods discussed herein may be configured to navigate and facilitate surgical procedures throughout the biliary apparatus 500. Of course, it should be appreciated that the apparatuses and methods discussed herein may be applied to a wide array of surgical procedures and anatomical settings.

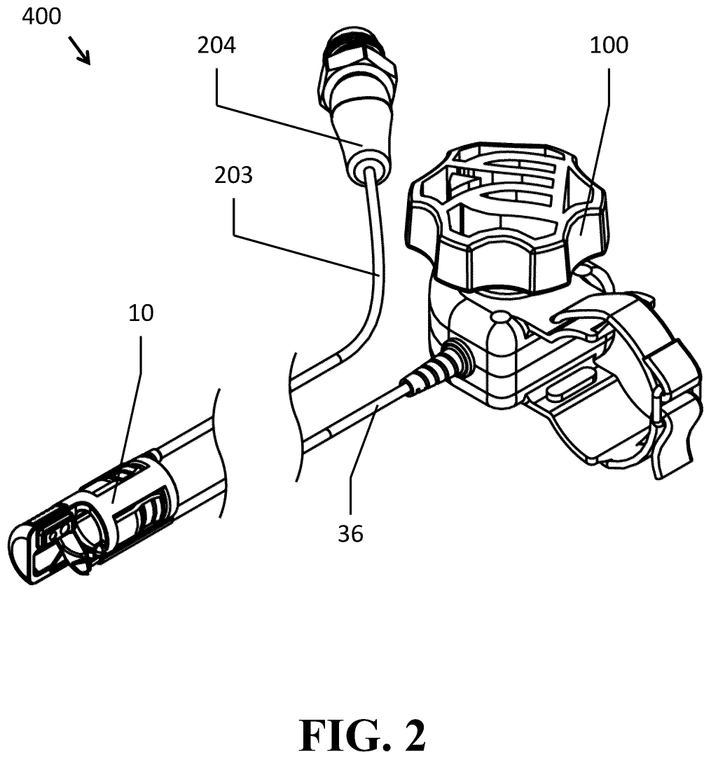

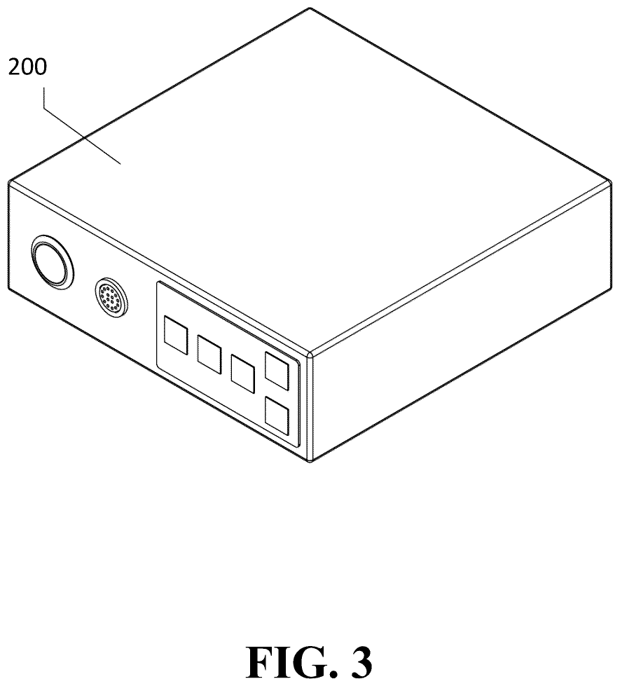

Referring now to FIGS. 2 and 3, an apparatus 400 is shown, according to some embodiments of the present disclosure. As discussed in greater detail below, the present disclosure may provide for a modular and removable endoscope apparatus 400, which may be configured for enabling digital visualization and cannulation of the minor or major duodenal ampulla. The apparatus 400 may include an endcap 10 and a user interface 100 (shown together with reference to FIG. 2), along with a control unit 200 (shown independently with reference to FIG. 3). As discussed in greater detail below, the endcap 10 may be coupled to the user interface 100 via a tube 36, and the endcap 10 may be coupled to the control unit 200 via a cable 203 and a connector 204.

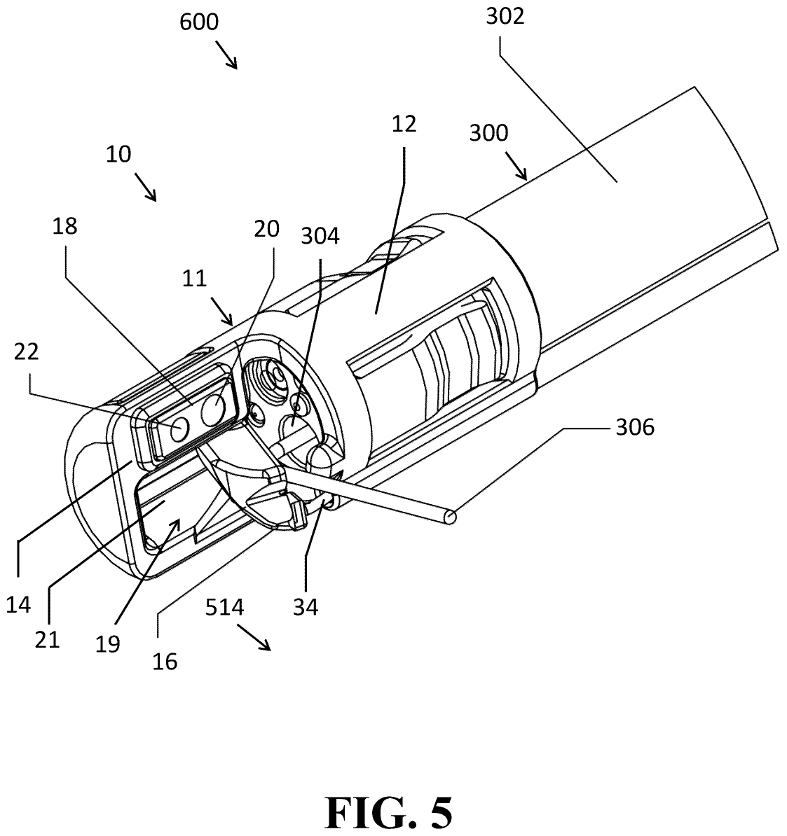

Referring now to FIG. 4, an apparatus 600 is shown, according to some embodiments of the present disclosure. In some embodiments, the apparatus 600 includes the apparatus 400 as discussed above, as well as the endoscope 300. The apparatus 400 may be removably integrated with the endoscope 300 in order to provide the apparatus 600. For instance, the user interface 100 may be removably secured to a handle 301 of the endoscope 300, and the endcap 10 may be removably secured to a distal end 302 of the endoscope 300 (as shown in greater detail with reference to FIG. 5). As discussed in greater detail below, the endoscope 300 may include a forward-facing working port 304 (e.g., a channel or passage formed in the body of the endoscope 300). A tool 306 may be configured to be advanced out of the working port 304.

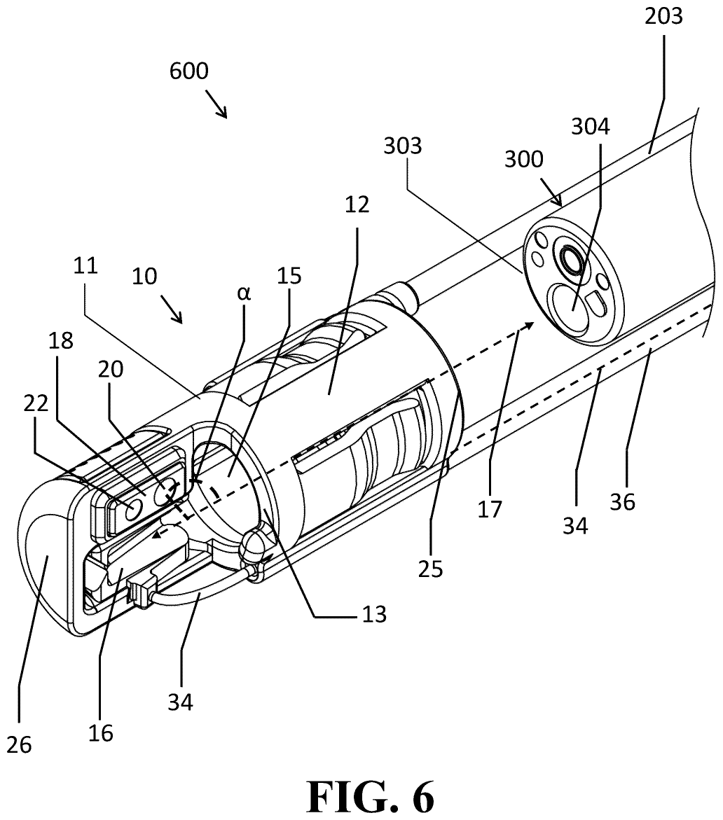

Referring now to FIGS. 5-6, the endcap 10 of the apparatus 600 is shown in greater detail, according to various embodiments of the present disclosure. The endcap 10 may include a body 11 and a side opening 19. The body 11 may be configured to fit onto the distal end 302 of the endoscope 300. Thus, the apparatus 400 (or, more generally, the apparatus 600) may include the endcap 10 including the body 11 and the side opening 19, which may be configured to fit onto the distal end 32 of the endoscope 300, where the endoscope 300 includes the forward-facing working port 304 configured for advancement of the tool 306 from the working port 304.

In some embodiments, the body 11 includes a collar portion 12 and a leading portion 14. For example, the body 11 may form a generally cylindrical shape extending from a proximal end 25 to a distal end 26 along a longitudinal axis 17 of the endcap 10. Such cylindrical shape may generally be conformed to by the collar portion 12. However, the leading portion 14 may form the side opening 19 on the cylindrical shape of the body 11, providing openings and exposed surfaces for facilitating the other components of the endcap 10 discussed herein. The side opening 19 may form a rim 13 on a distal end of the collar portion 12 where the collar portion 12 and the leading portion 14 join along the longitudinal axis 17 of the body 11. The rim 13 may partially surround an intermediate opening 15 formed on the distal end of the collar portion 12. As discussed in greater detail below, components of the endoscope 300 may be extended through the intermediate opening 15, such that the tool 306 may be advanced out of the working port 304.

The collar portion 12 may be removably secured to the distal end 302 of the endoscope 300, and the leading portion 14 may extend from the collar portion 12 and away from the distal end 302 of the endoscope 300. In this sense, the collar portion 12 (and thus the endcap 10 in its entirety) may be mechanically attached to the distal end 302 of the endoscope 300 through a removable mechanical fixation method that is non-damaging to the endoscope 300 and the endcap 10.

Depending on the implementation, some or all of the components of the endcap 10 may be made of any suitable medical-grade, biocompatible material, including but not limited to thermoplastics such as Acrylonitrile Butadiene Styrene (ABS) and Polycarbonate (PC), or metals such as stainless steel and titanium. Further, some or all components of the endcap 10 may be manufactured from injection molding or machining.

In some embodiments, the endcap includes an elevator 16 pivotably coupled to the body 11. For instance, the elevator 16 may be a lever, an arm, etc. The elevator 16 may be pivotably coupled to the body 11 of the endcap and, as discussed in greater detail below, pivoted relative to the body 11.

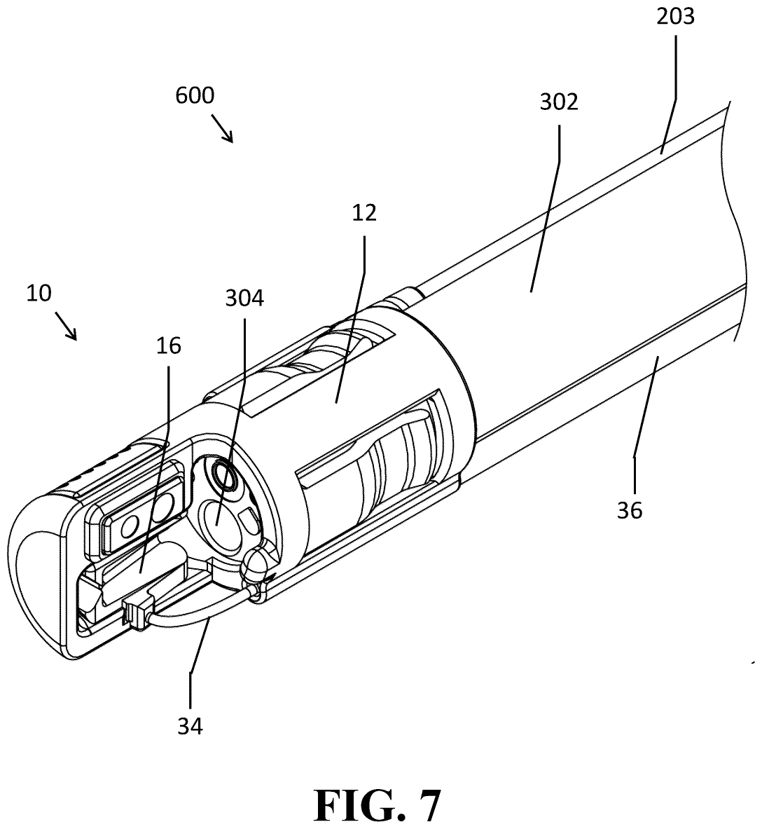

In some embodiments, the elevator 16 is an articulating mechanism disposed in a cavity 21 formed on an interior of the body 11 of the endcap 10 (e.g., a surface of the leading portion 14 of the body 11 which faces the side opening 19). As discussed in greater detail below with reference to FIGS. 7-10, the elevator 16 may be pivoted relative to the body 11 of the endcap 10 such that the elevator 16 deflects the tool 306 towards the side opening 19 of the endcap 10 into a lateral workspace 514 near the endcap 10 (e.g., a workspace that is lateral to both the endcap 10 and the distal end 302 of the endoscope 300). For instance, the elevator 16 may be configured to direct (e.g., bend, deflect, re-orient, etc.) the tool 306 towards or about a surgical site. Depending on the implementation, the tool 306 may include guidewires, catheters, cannulas, sphincterotomes, stents, balloons, baskets, and so on. Accordingly, the apparatus 400 (or, more generally, the apparatus 600) may include the elevator 16 coupled to the body 11 of the endcap 10, which may be configured to be pivoted relative to the body 11 such that the elevator 16 deflects the tool 06 towards the side opening 19 of the endcap 10 into the workspace, such as the lateral workspace 514.

In some embodiments, the endcap 10 includes a visualization and illumination assembly 18. The visualization and illumination assembly 18 may include an image sensor 20 disposed on the leading portion 14 and a light source 22 disposed on the leading portion 14. The visualization and illumination assembly 18 may be configured for visualizing and recording the surgical area (including, but not limited to, the lateral workspace 514) such that the anatomy and the heading of the tools 306 can be monitored. Accordingly, the apparatus 400 (or, more generally, the apparatus 600) may include the image sensor 20 disposed on the body 11 of the endcap 10, which may be configured to visualize a workspace such as the lateral workspace 514.

As suggested above, the endcap 10 may include the image sensor 20 disposed on the body 11. The image sensor 20 may be configured to visualize the lateral workspace 514. In particular, the image sensor 20 may be positioned on an interior of the body 11 of the endcap 10, such that the image sensor 20 visualizes the lateral workspace 514 through the side opening 19 of the endcap 10. In some embodiments, the image sensor 20 is a complementary metal-oxide semiconductor (CMOS) image sensor unit integrated into the sub-assembly. In other words, the image sensor 20 may include a CMOS sensing array. The image sensor 20 may include requisite optical components (lenses, filters, etc.) in addition to a CMOS sensing array of the CMOS image sensor.

The image sensor 20 may be configured to provide sufficient native resolution to perform high-quality imaging of the surgical field. Such resolution may reside between 100×100 pixels to 3840×2160 pixels, and the field of view of the image sensor 20 may be between 60 degrees and 180 degrees (relative to the longitudinal axis 17 of the body 11) to offer optimal visual coverage of the duodenal papilla. Such resolution may be optionally up-sampled in software (stored on the video process 200) to realize a higher effective resolution via interpolation algorithms. The image sensor 20 may be singular and monoscopic in nature. In other embodiments, the visualization and illumination assembly 18 includes two image sensors 20 (e.g., two CMOS image sensors) placed side-by-side to implement stereoscopic imaging.

The image sensor 20 may be configured such that it is compatible with a desired sterilization method associated with the endoscopic procedure, which may include gamma radiation, ethylene oxide (ETO), or electron beam (E-Beam) sterilization, among others. In some embodiments, the image sensor 20 is coated with an anti-fouling or anti-fogging film to prevent bodily fluid and debris from fouling a lens of the image sensor 20. In the event of lens fouling, the image sensor 20 may be positioned such that the image sensor 20 can be cleaned via irrigant flow from an auxiliary water channel of the endoscope 300. In some embodiments, the endcap 10 itself includes an irrigation channel that is configured to clean off the image sensor 20 when such an irrigation channel is in communication with an external irrigation source at the proximal end of the device (e.g., the end of the apparatus 600 formed by the opposite end of the endoscope 300, handle 301 and/or the user interface 100).

As suggested above, the endcap 10 may include the light source 22 disposed on the body 11. The light source 22 may be configured to illuminate the lateral workspace 514 or, more generally, the surgical field. For example, the light source 22 may include a light-emitting diode (LED) disposed on the leading portion 14 of the endcap 10, alongside the image sensor 20. In other embodiments, the light source 22 is an optical fiber bundle which terminates distally within the endcap 10, and proximally at an illumination source such as an off-board LED or incandescent bulb (e.g., stored on the control unit 200 or the user interface 100) for the purpose of transmitting light generated from the off-board optical light source to the surgical field. As shown, the light source 22 may be disposed on the leading portion 14 of the endcap 10 adjacent to the image sensor 20 such that the field of view of the image sensor 20 and the illumination field provided by the light source 22 overlap.

In some embodiments, the visualization and illumination assembly 18 is oriented such that the axis of visualization for the image sensor 20 is at an angle with respect to the longitudinal axis 17 of the endcap and, therefore, the endoscope 300, to enable lateral visualization of a lumenal wall within the lateral workspace 514. Such an axis of visualization may form an angle a of between 0 degrees and 135 degrees with respect to the longitudinal axis 17 of the endoscope 10. Depending on the implementation, the angle a may be selected to maximize visualization of the duodenal papilla when the distal end 302 of the endoscope 300 is positioned in the proximity of the papilla. Further, the angle a may be selected such that the tools 306 of the endoscope 300 are brought into view of the image sensor 20 when the elevator 16 of the endcap 10 is articulated.

In some embodiments, the visualization and illumination assembly 18 is housed within an injection-molded component that is directly molded into the body 11, or assembled into the body 11 via adhesive bonding, snap-fit assembly, or ultrasonic/radio-frequency welding. In other embodiments, the visualization and illumination assembly 18 is non-destructively separable from the body 11 to enable separate sterilization and re-use.

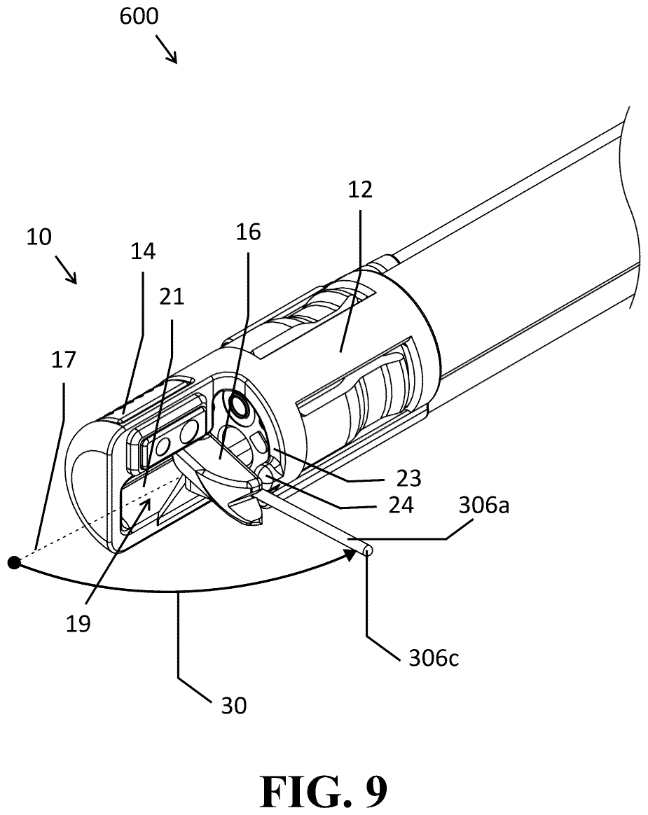

Referring now to FIGS. 7-10, the elevator 16 of the endcap 10 is discussed in greater detail, according to some embodiments of the present disclosure. Generally, the elevator 16 may be positioned at least partially within the cavity 21 formed by the body 11 of the endcap 10. As mentioned above, the elevator 16 may be pivotably coupled to the body 11. In this sense, the elevator 16 may be coupled to the body 11 via a rotary joint 38 disposed within the endcap 10. Accordingly, the elevator 16 may be pivoted relative to the body 11 between (and including) a first position, which may be considered a neutral orientation (as shown with reference to FIG. 7), where the elevator 16 is oriented substantially parallel to the longitudinal axis 17 of the endcap 10, and a second position, which may be considered an engaged orientation (as shown with reference to FIG. 9), where the elevator is oriented towards the lateral workspace 514, and any position therebetween. When the elevator 16 is positioned in the neutral orientation, the elevator 16 may be positioned at least partially within the cavity 21 formed on the leading portion 14 of the body 11 of the endcap 10, leaving the area around the working port 304 of the endoscope 300 unobstructed and allowing for the advancement of the tool 306 out of the working port 304.

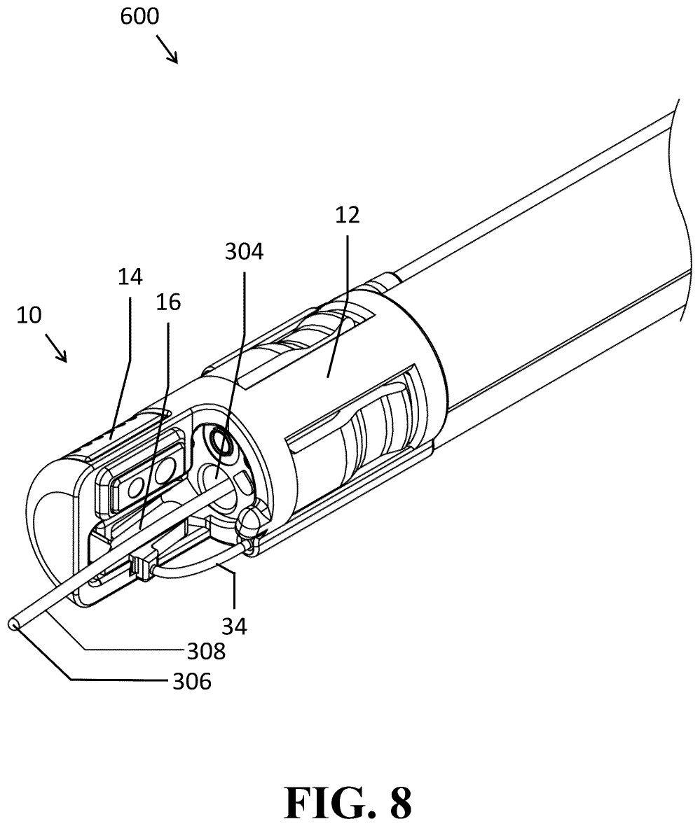

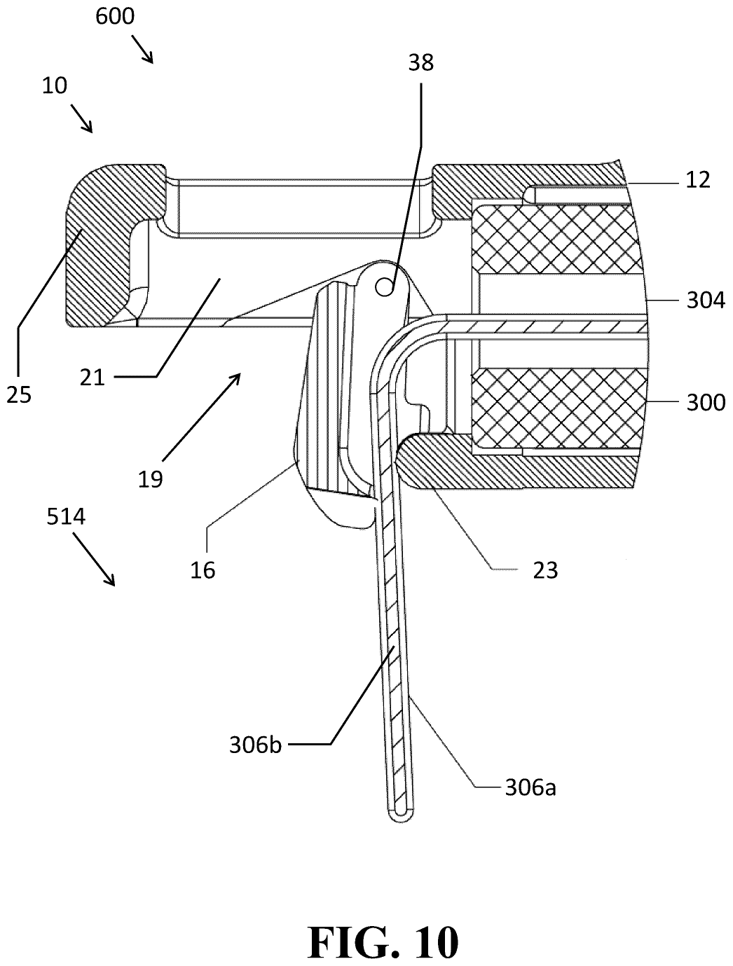

As suggested above, the elevator 16 may be configured to align with the working port 304 of the endoscope 300. For instance, the working port 304 may terminate at the distal tip 303 of the endoscope 300, over which the intermediate opening 15 of the collar portion 14 (shown with particular reference to FIG. 6) may be aligned when the endcap 10 is secured to the endoscope 300. As shown with particular reference to FIG. 8, the tool 306 may be advanced out of the working port 304 until the tool 306 extends into (or past) the leading portion 14 of the endcap 10. Once the tool 306 has been extended out of the working port 304, and as shown with particular reference to FIG. 9, the elevator 16 may be pivoted relative to the body 11 of the endcap 10, making contact with the tool 306 and bending the tool 306 along a path 30 towards a desirable location within the surgical site (including, but not limited to, a location within the lateral workspace 514). For instance, FIG. 8 depicts the elevator 16 in the neutral orientation, while FIGS. 9 and 10 depict the elevator 16 in the engaged orientation. Of course, it should be appreciated that the elevator 16 may be pivoted to any position in between the depicted unengaged and engaged orientations. Accordingly, the elevator 16 may be configured to mechanically engage with the tool 306 passed through the working port 304 of the endoscope 300, deflecting the tools 306 laterally. In this sense, the elevator 16 may be pivoted relative to the body 11 of the endoscope 10, such that that elevator 16 deflects the tool 306 toward the side opening 19 of the endcap into the lateral workspace 514.

In some embodiments, and as shown with particular reference to FIGS. 9 and 10, the tool 306 includes a guidewire 306a forming an open channel 306b therein, through which a tool element 306c can be extended into and protruded from. Depending on the implementation, the endcap 10 may include a mechanism for “locking” the guidewire 306a from further advancement or retraction when the tool element 306c is passed through the open channel 306b. Advantageously, instead of requiring the user to manually lock and reposition a portion of the guidewire 306a that protrudes from the endoscope 300 as tools are inserted over the guidewire 306a, as is typical in conventional “long-wire” systems, the present disclosure provides for a method of “locking” the guidewire 306a near a distal end of the guidewire 306a via the elevator 16, thereby holding the guidewire 306a in place as tool(s) 306c are passed through the open channel 306b. In this sense, the elevator 16 may be configured to deflect the tool 306 until the tool 306 is secured in place between the elevator 16 and an edge of the side opening 19 of the endcap 10.

In the conventional “long wire” approach to tool exchange, a very long guidewire (which is, at a minimum, at least twice as long as the endoscope) is first inserted through the endoscope to cannulate the ampulla. Once cannulation is achieved, approximately half of the guidewire's length protrudes from the endoscope's tool channel inlet at the proximal end, allowing the user to grasp the guidewire while sliding another tool over the proximal end of the guidewire to prevent the guidewire from migrating or moving as the tool is slid over it. Once the tool is advanced over the distal end of the guidewire to the entrance of the working channel inlet, the user repositions his or her grasp to hold the very proximal end of the guidewire which is now protruding from the proximal end of the tool. With the guidewire mechanically fixed by the user, it is held in place, and the tool can be fully advanced into the endoscope over the guidewire, and ultimately out of the distal end of the endoscope.

This approach discussed herein may be preferable over the “long-wire” in clinical settings, as longer guidewires associated with manual manipulation can be unwieldy and difficult to work with due to their length. Additionally, the use of shorter guidewires allowed by the mechanical locking discussed herein can reduce instances of inadvertent contact with non-sterile surfaces that otherwise may be a concern with longer guidewires of conventional systems.

In some embodiments, and as an example of the mechanical locking of shorter guidewires discussed above, when the elevator 16 is actuated (e.g., pivoted towards the engaged orientation as shown with reference to FIG. 9), the guidewire 306a becomes “sandwiched” between the elevator 16 and a locking surface 23 on the body 11 of the endcap 10. For instance, and as mentioned above, the side opening 19 of the leading portion 14 may form the rim 13 on a distal end of the collar portion 12. In some embodiments, the locking surface 23 is a groove (e.g., a notch, knurl, bump, or similar surface) that projects from the rim 13 into the side opening 19 (e.g., away, relative to the longitudinal axis 17, from the proximal end 25 shown with reference to FIG. 6). In other embodiments, the locking surface 23 is the rim 13 itself.

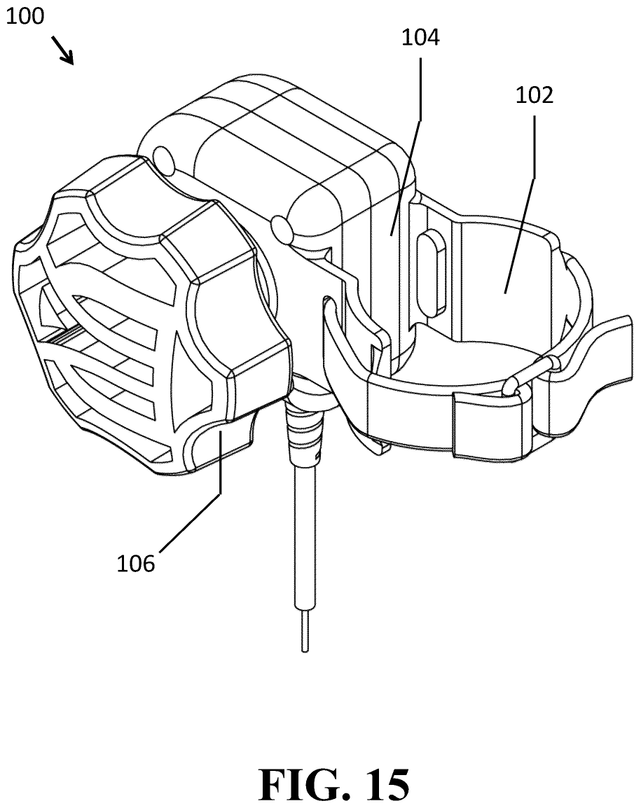

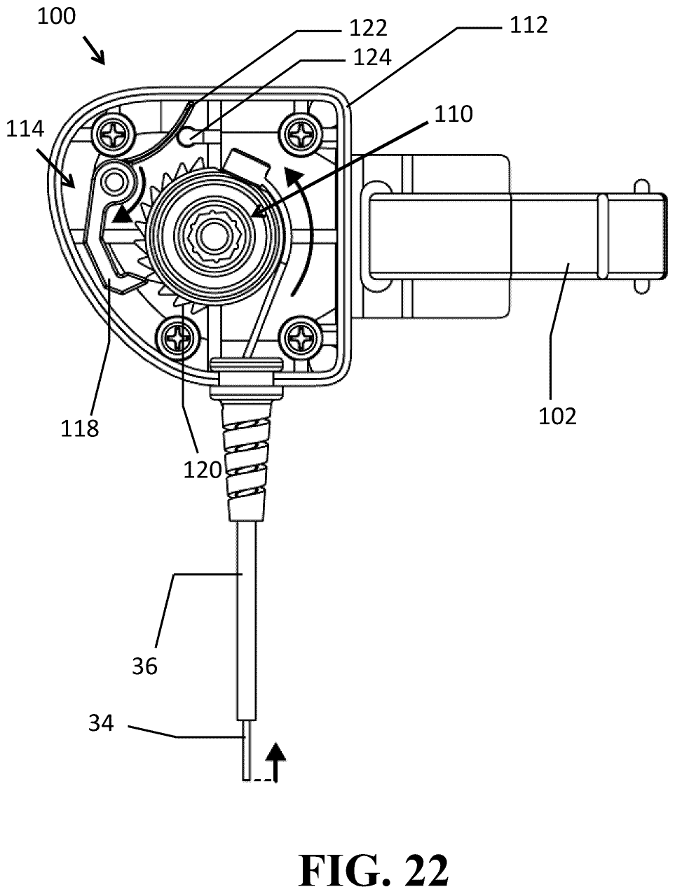

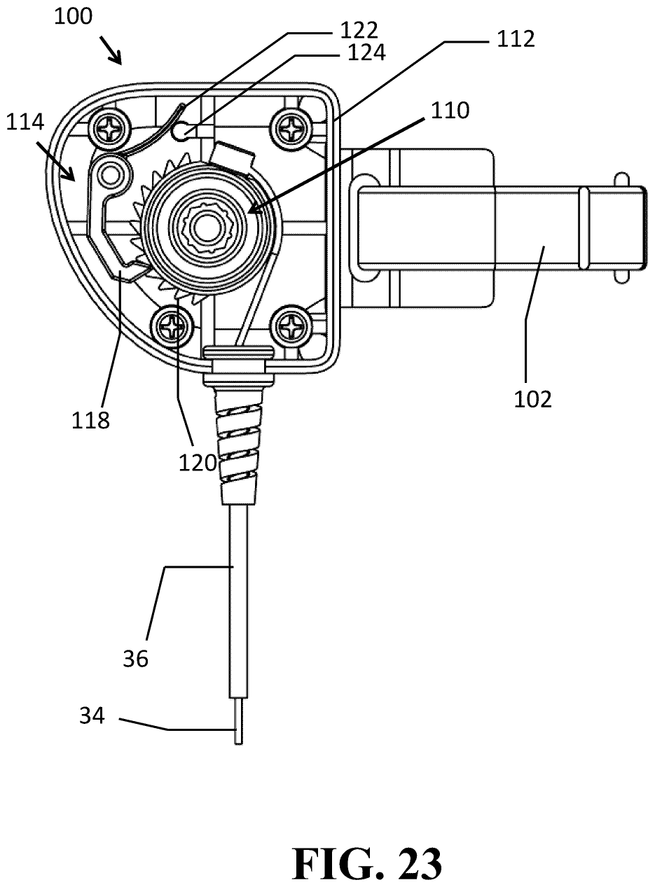

When the elevator 16 is in the engaged orientation as shown with reference to FIG. 9, the locking surface 23 may engage with a corresponding surface on the elevator 16 to pinch the guidewire 306a. The frictional force generated by such pinching may prevent the guidewire 306a from migrating as the tool element 306c is being advanced or retracted through the open channel 306b of the guidewire 306a. Thus, when the tool element 306c is secured in place between the elevator 16 and the aforementioned edge of the side opening 19, the tool element 306c may resist advancement from or retraction into the working port 304. As described in greater detail with reference to FIGS. 15-26, the deflection of the elevator 16 can be locked relative to the endcap 10.

Referring again to FIG. 6, in some embodiments, the elevator 16 is externally actuated (e.g., pivoted relative to the body 11 of the endcap 10 to one or more positions between, and including, the neutral orientation depicted with reference to FIG. 8, and the engaged orientation depicted with reference to FIGS. 9 and 10) by a pull-wire 34 (or a number of pull-wires) coupled to user interface 10 and the elevator 16. The pull-wire 34 may be housed within the tube 36. In some embodiments, the user interface 10 is configured to displace the pull-wire 34. Thus, the elevator 16 may be configured to be pivoted relative to the body 11 of the endcap 10 in response to displacement of the pull-wire 34.

Depending on the implementation, the pull-wire 34 may be made of any material, and provided in any configuration, suitable to apply a sufficient tensile force to “pull” the elevator when the pull-wire 34 is displaced towards the user interface 10, such that the elevator 16 is pivoted towards the engaged orientation. Further, the pull-wire 34 may feature sufficient axial stiffness to enable it to “push” the elevator 16 when the pull-wire 34 is displaced away from the user interface 10, such that the elevator 16 is pivoted towards the neutral position. The pull-wire 34 may be made of any suitable material and provided in any suitable configuration, including but not limited to wire (e.g., a single wire) or braided rope configurations, stainless steel, nitinol, and Kevlar materials, and so on. In this sense, the elevator 16 may be configured to be pivoted towards the working port 304 of the endoscope 300 when the pull-wire 34 is displaced towards the user interface 10, and the elevator 16 may be configured to be pivoted away from the working port 304 when the pull-wire 34 is displaced away from the user interface 10.

Accordingly, when the aforementioned tensile force is applied to the pull-wire 34, the elevator 16 is configured to pivot about the rotary joint 38 (depicted with reference to FIG. 10) disposed within the endcap 10 to rotate out of the cavity 21 in the direction of the tool 306 and mechanically engage with the tool 306, applying a lateral force to the tool 306, which may cause the tool 306 to deflect, as depicted with reference to FIG. 9. The amount of deflection imparted on the tool 306 may be controlled by the amount of the aforementioned tension applied to the pull-wire 34, which may change the deflection angle of the elevator 16, thereby allowing a user to controllably affect the angle of the tool 306 with respect to the longitudinal axis 17 of the endcap 10.

The tool 306 may feature sufficient elasticity to allow the tool 306 to passively straighten when the elevator 16 is pivoted towards the neutral orientation (e.g., the deflecting force otherwise on the tool 306 by the elevator 16 is removed). The elevator 16 may be made of any suitable material and made from any suitable manufacturing process, including but not limited to being injection-molded from a suitable biocompatible thermoplastic. Depending on the implementation, the elevator 16 may be constructed from or coated with a lubricious material to facilitate passage of the tool 306 over a surface of the elevator 16 when the elevator 16 is in any position including and between the neutral orientation depicted with reference to FIG. 8, and the engaged orientation depicted with reference to FIGS. 9 and 10). In some embodiments, the elevator 16 is machined from a biocompatible metal such as stainless steel or titanium. The elevator 16 may include curvilinear surface molded or machined into it for the purpose of re-directing the tool 306 laterally as discussed herein.

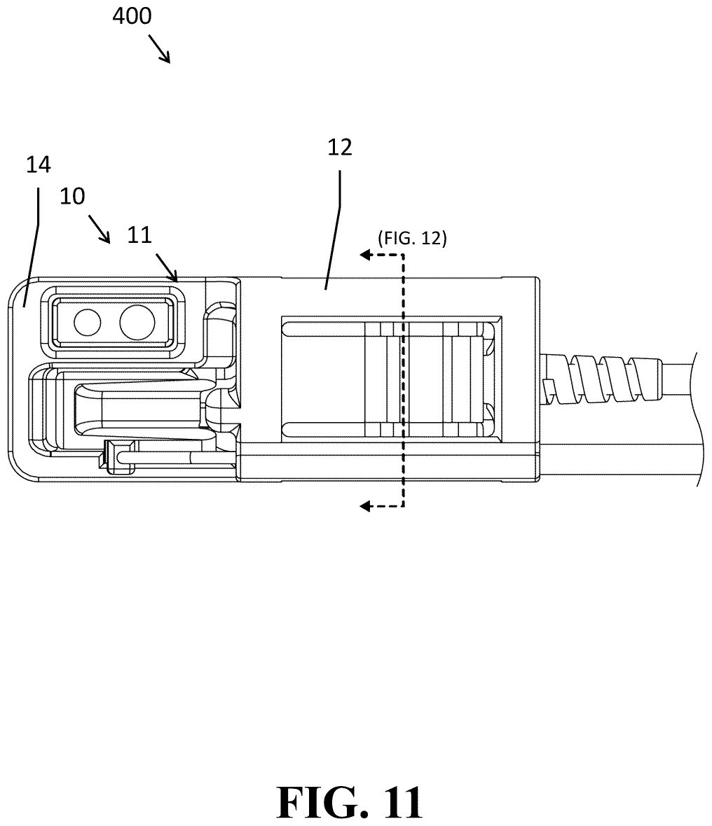

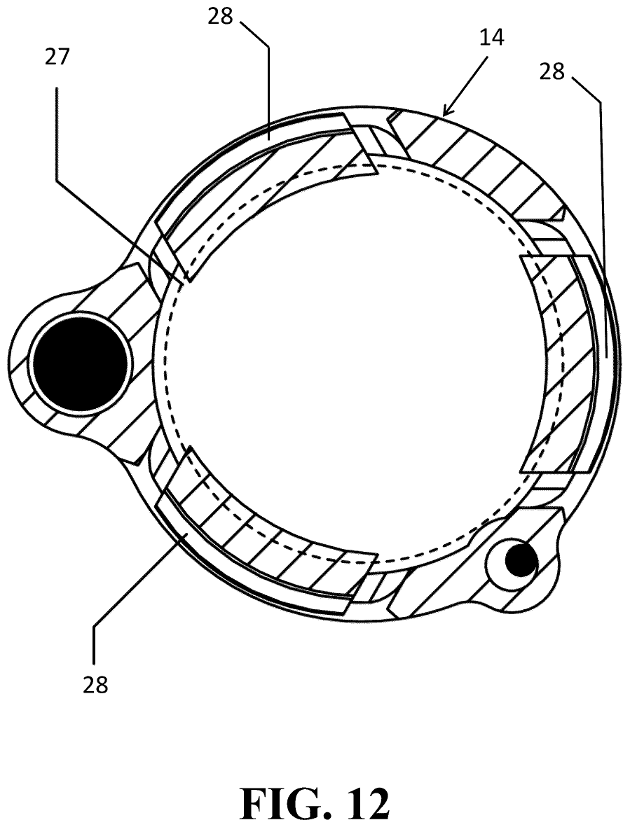

As mentioned above, the collar portion 12 (and thus the endcap 10 in its entirety) may be mechanically attached to the distal end 302 of the endoscope 300 through a removable mechanical fixation method that is non-damaging to the endoscope 300 and the endcap 10). Referring now to FIGS. 11 and 12, such mechanical fixation may be achieved through a series of flexural elements 28, according to some embodiments of the present disclosure. The flexural elements 28 may be distributed around a circumference 27 of the collar portion 14 of the body 11 of the endcap 10. When the endcap 10 is disposed on the distal end 302 of the endoscope 300, the flexural elements 28 may be configured to elastically deflect outwards radially (e.g., away from the longitudinal axis 17 of the body 11 shown with reference to FIG. 6), thereby providing a reactionary preload spring force radially inward against the endoscope 300 to affix the endcap 10 to the endoscope 300.

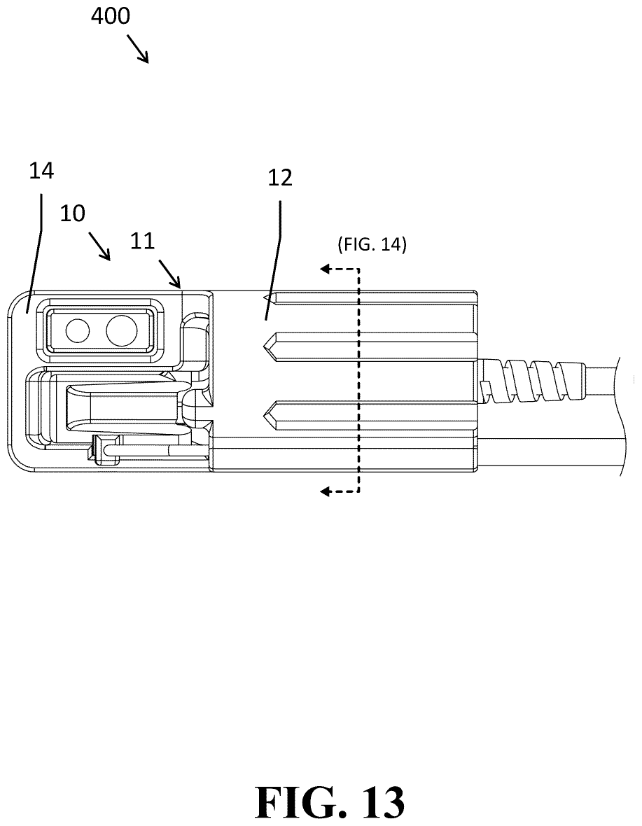

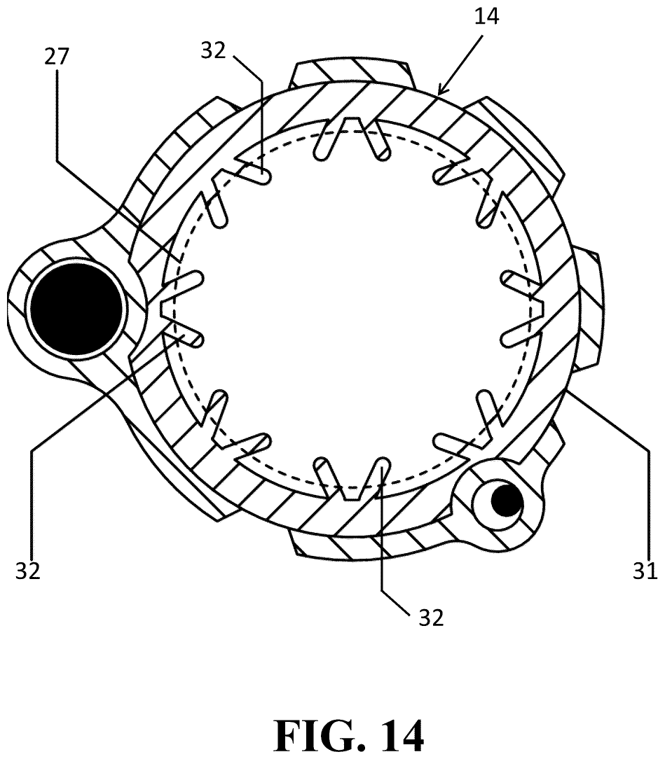

Referring now to FIGS. 13 and 14, such mechanical fixation may be achieved through the incorporation of a ring 31 of low-stiffness, low-durometer material, according to other embodiments of the present disclosure. The ring 31 may be made of any suitable material including, but not limited to, thermoplastic elastomer or rubber. The ring 31 may project into the circumference 27 of the collar portion 14, thereby creating a high-friction interface between the body 11 of the endcap 10 and the endoscope 300. For example, the ring 31 may form a series of crush ribs 32 which deform when the endoscope 300 is pressed into the endcap 10 to create a high-friction seal.

Referring now to FIG. 15, the user interface 100 is shown in greater detail, according to some embodiments of the present disclosure. As mentioned above, the user interface 100 may be removably secured to a handle 301 of the endoscope 300. For instance, the endoscope 300 and the handle 301 thereof may be a third-party endoscope. In some embodiments, the user interface 100 is removably secured to the handle 301 by a strap 102. The strap 102 may be tightened around the handle 301 and be made of any suitable material including, but not limited to, rubber or hook and loop. In other embodiments, the user interface 102 is secured to the handle 301 by a thumbscrew that can be tightened against the handle 301. In other embodiments still, the user interface 102 is secured to the handle 301 by a snap-fit interface. For example, such snap-fit interface may include flexural elements on the user interface 100 that mechanically engage with features on the handle 301. The user interface 100 may feature a transmission 104 (e.g., a transmission sub-assembly) that enables a user to apply sufficient tension to the pull-wire 34 in order to pivot the elevator 16 as discussed above.

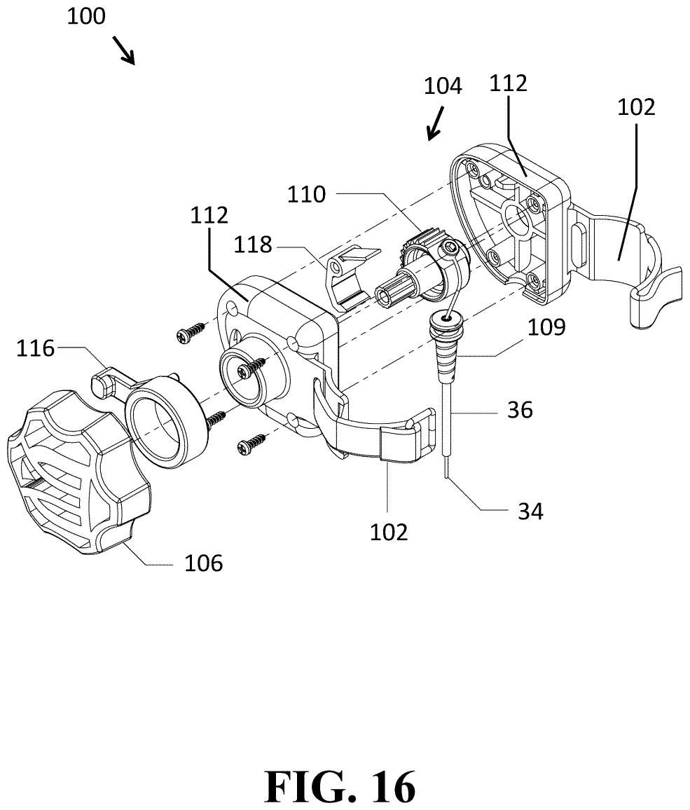

Referring now to FIGS. 16-20, the transmission 104 of the user interface 100 is shown in greater detail, according to some embodiments of the present disclosure. A knob 106 (e.g., a rotatable knob, an elevator knob, etc.) transmits a user-applied rotation into pivoting the elevator 16 via a capstan 110 and the pull-wire 34. The pull-wire 34 may be coupled to the elevator 16 as discussed above, and be routed from the elevator 16 within the endcap 10 to the user interface 100 via the tube 36 (e.g., a Bowden cable-in-sheath). The tube 36 may be constructed from any suitable material, including but not limited to biocompatible thermoplastics, thermosets, or fluoropolymers, such as PEEK, PEBAX, Nylon 12, Polyimide, PTFE, HDPE, and so on. The tube 36 may be provided in any suitable configuration, including but not limited to a composite construction. The tube 36 may include a lubricious inner liner (for example, PTFE), a braid or coil layer (for example, stainless steel), an optional tie layer, and/or an outer jacket layer.

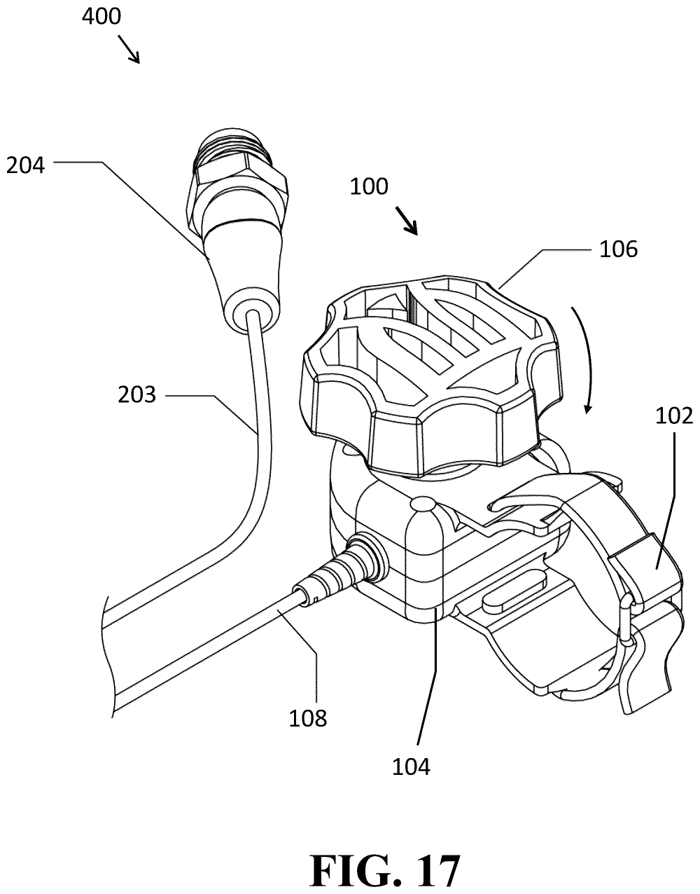

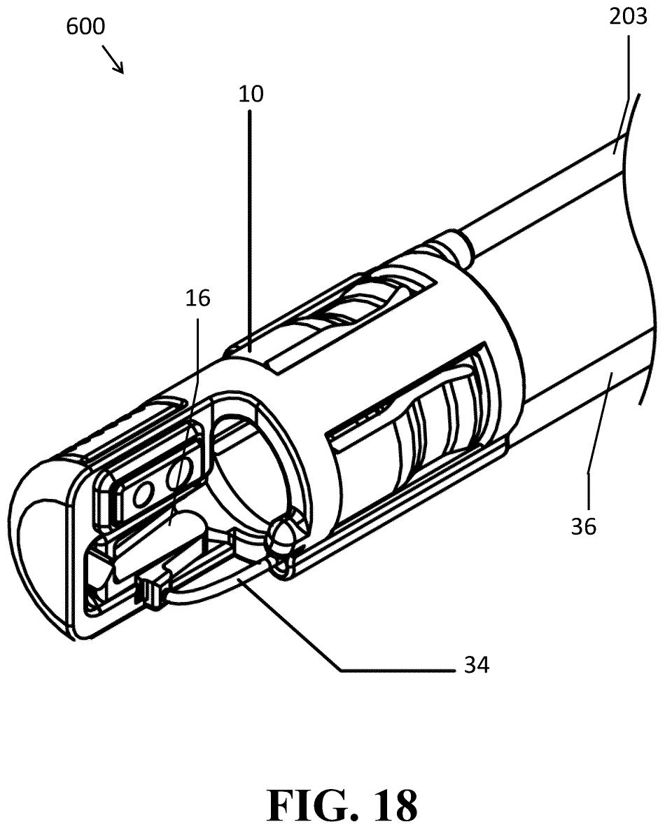

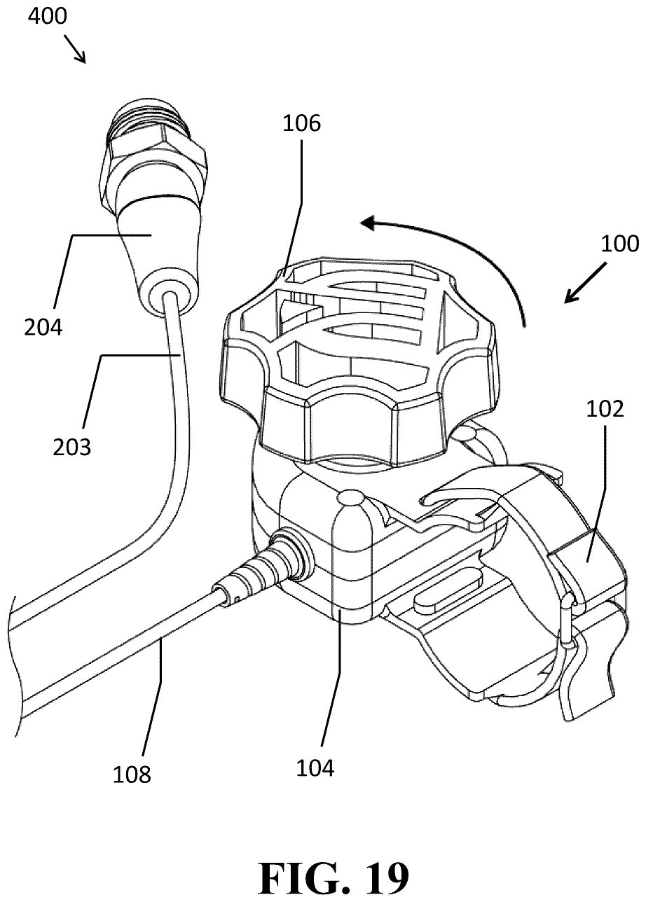

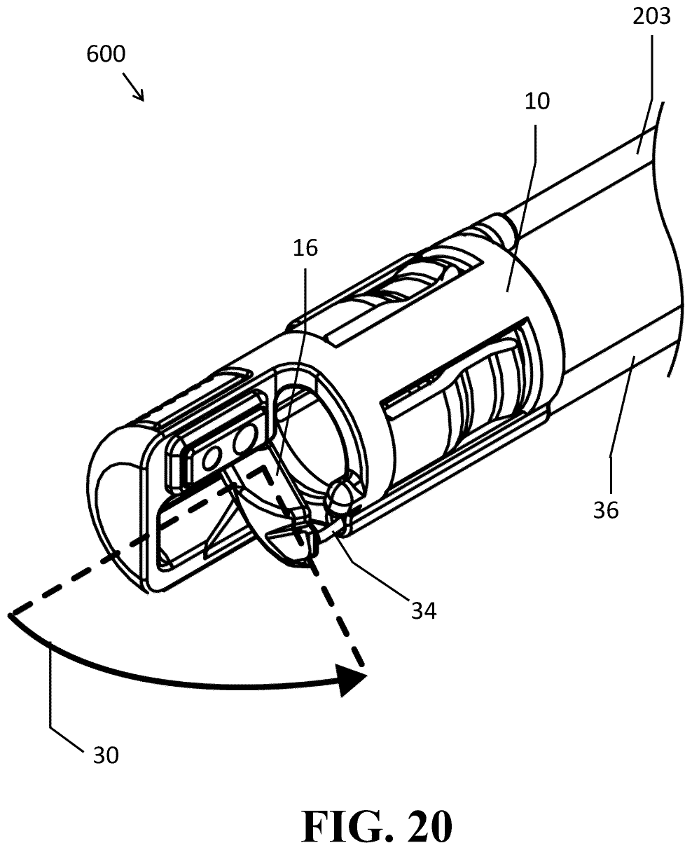

In some embodiments, the pull-wire 34 is nested inside the tube 36 and is configured to move axially with respect to the tube 36. The pull-wire 34 may exit the tube 36 as the pull-wire 34 enters into the user interface 100 and may be coupled to the capstan 110, which may be coupled to the knob 106. As shown with particular reference to FIGS. 19-20, as the user rotates the knob 106 in one direction (e.g., counter-clock-wise), the coupled rotation of the capstan 110 may apply tension in the pull-wire 32, which may result in pivoting of the elevator 16 towards the engaged orientation, as depicted with reference to FIG. 9. As shown with particular reference to FIGS. 17-18, by rotating the knob 106 in the other direction (e.g., clockwise), the tension is relaxed in the pull-wire 34 and the elevator 16 may be pivoted towards the neutral orientation. As examples, such pivoting due to relaxation of the tension in the pull-wire 34 may be a result of elasticity of the pull-wire 34, elasticity of the tool 306 being deflected by the elevator 16, or through axial force applied to the elevator 16 by the pull-wire 34 itself. In other embodiments, a leadscrew transmission is used to convert user input motion into pull-wire 34 tension. In other embodiments still, a rack-and-pinion transmission is used, where the knob 106 drives a pinion gear which engages with a rack that is coupled to the pull-wire 34.

In some embodiments, the user interface 100 includes a housing 112. The housing 112 may be provided in any suitable configuration, including but not limited to a two-part construction made from medical-grade, injection-moldable plastic such as ABS or PC. In some embodiments, the user interface 100 includes a strain relieving component 109 on the tube 36 where the tube 36 exits the housing 112. The strain relieving component 109 may be provided in any suitable configuration, including but not limited to a construction from injection-moldable silicone rubber or thermoplastic elastomer.

In some embodiments, the transmission 104 includes ratcheting or a lock 114 (e.g., a locking mechanism, a locking assembly, etc.) that maintains tension in the pull-wire 34 when the knob 106 or other input control feature is released by the user. The lock 114 may be useful to maintain an orientation of the elevator 16 (e.g., an orientation including or between the unengaged and engaged orientations) and, thereby, the tool 306 being deflected by the elevator 16 without requiring the user to continuously apply an actuation force to knob 106, thereby freeing up his or her hand for other tasks. In some embodiments, the lock 114 includes a lever 116 (e.g., a lock lever) and a pawl 118. As discussed in greater detail below, the lock 114 may allow the user to take his or her handle off of the knob 106 without the elevator 16 returning to its neutral orientation (and, thereby, relieving the frictional force between the tool 306 and the elevator 16 and/or locking surface 23).

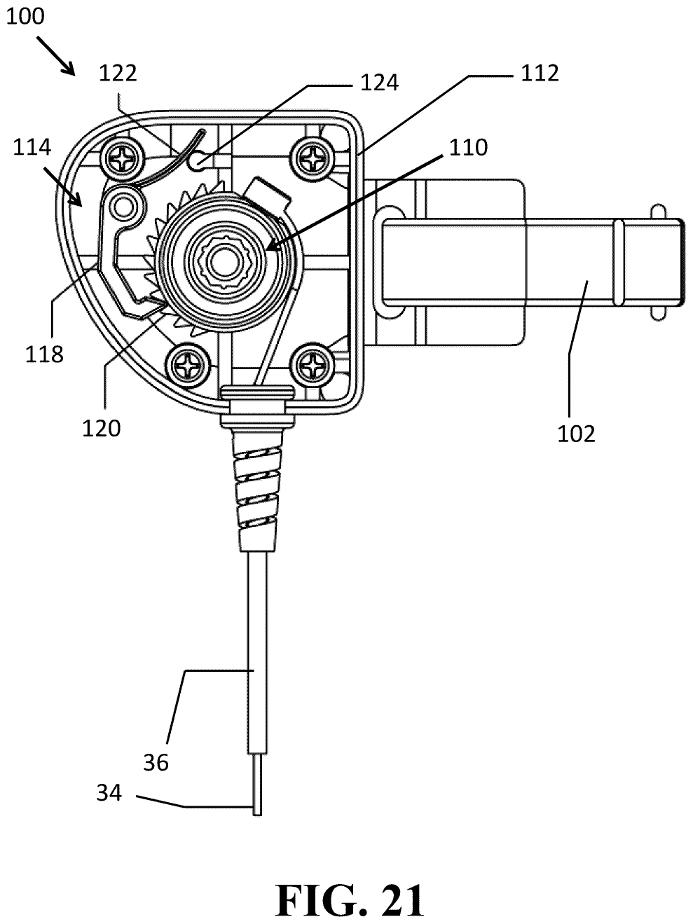

Referring now to FIGS. 21-25, the lock 114 is shown in greater detail, according to some embodiments of the present disclosure. As the knob 106 is twisted counter-clockwise (as shown with reference to FIGS. 19-20), establishing a tension in the pull-wire 34, a ratchet 120 (e.g., a ratcheting surface) formed on the capstan 110 engages with the pawl 118, which may allow for free rotation of the capstan 110 in the counter-clockwise direction, but prevent clockwise rotation of the capstan 110. In some embodiments, the pawl 118 is spring-loaded in order to retain the capstan 110 in a “closed” configuration where free rotation in the counter-clockwise direction is allowed, by clockwise rotation is prevented. For example, a spring force may be provided by a leaf spring 122 formed on the pawl 118, which may engage with a stop 124 (e.g., a hard mechanical stop) that is formed on the housing 112 of the user interface 110. In other embodiments, this spring force may be provided by a torsion, tension, or compression spring. Such a feature may maintain tension in the pull-wire 34 when the driving torque is removed from the control knob 106, as the engagement between the ratchet 120 and the pawl 118 prevents the capstan 110 from rotating backwards.

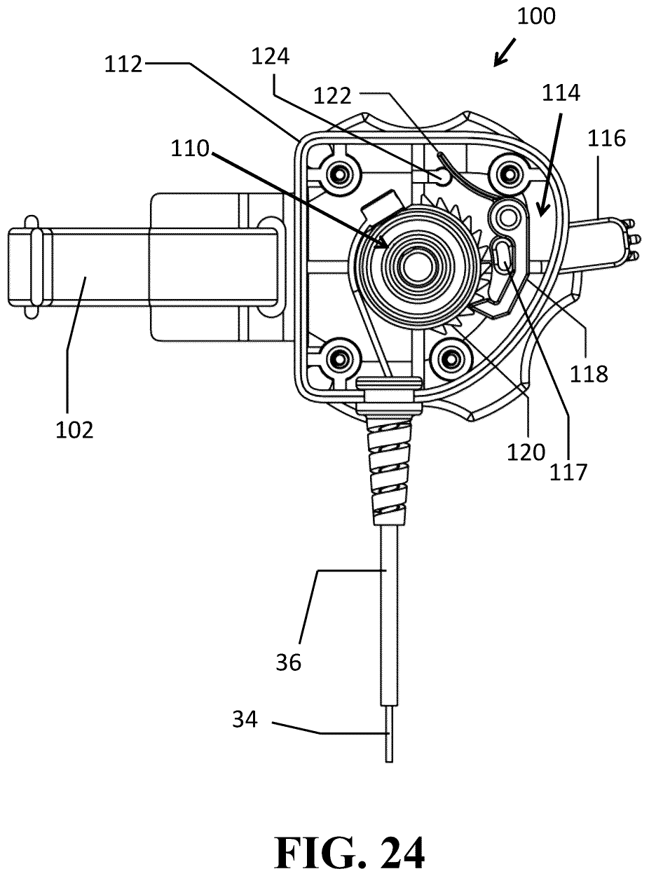

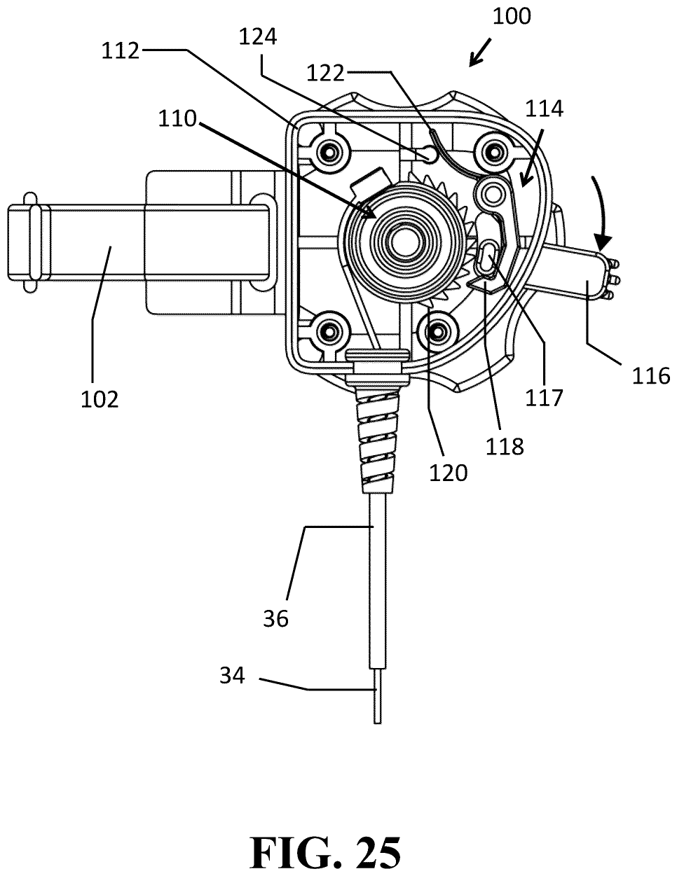

In some embodiments, in order to disengage the pawl 118 from the ratchet 120 and allow for both clockwise and counter-clockwise rotation of the capstan 110 (thereby adjusting the capstan 110 to an “open” configuration), the lever 116 may be rotated as shown in FIGS. 24 and 25. For example, the lever 116 may include a tab 117 that extends into the housing 112 and engages with a cam surface on the pawl 118. As the lever 116 is rotated down in the clockwise direction, the engagement between the tab 117 and cam surface of the pawl 118 may forces the pawl 118 to pivot away from the ratchet 120 on the capstan 110 (e.g., against the biasing force of the leaf spring 122), thereby allowing the capstan 110 to rotate freely. In this sense, the leaf spring 122, when coupled with the geometry of the cam surface of the pawl 118 and the tab 117 of the lever 116, creates a bistable mechanism such that the lever 116 experiences mechanical equilibrium in both the open and closed configurations of the capstan 110.

In further embodiments, instead of a ratchet/pawl interface which wholly prevents backwards (e.g., clockwise) rotation of the capstan 110 when the pawl 118 is engaged as discussed above, the lock 114 may be provided via a friction brake mechanism. In such embodiments, a high-friction pad on the pawl 118 is pre-loaded against the surface of the capstan 110 surface via the leaf spring 122, or a user-controllable thumbscrew, in order to provide resistance against rotation of the capstan 110. As examples, the high-friction pad may be constructed of a low-durometer material, such as silicone rubber or thermoplastic elastomer, which may be over-molded onto the pawl 118. A material stiffness of the leaf spring 122 may determine an amount of frictional force that the pawl 118 applies to the capstan 110 to resist rotation of the capstan 110. The aforementioned amount of frictional force may be configured such that suitable tension in the elevator 16 is maintained when the knob 106 is in a certain position, but the user can “overcome” the frictional force with a deliberate rotation of the knob 106. In other embodiments, the user-controllable thumbscrew may be used to tighten the pawl against the capstan, allowing the user to precisely configure the amount of frictional force by tightening or loosening the thumbscrew.

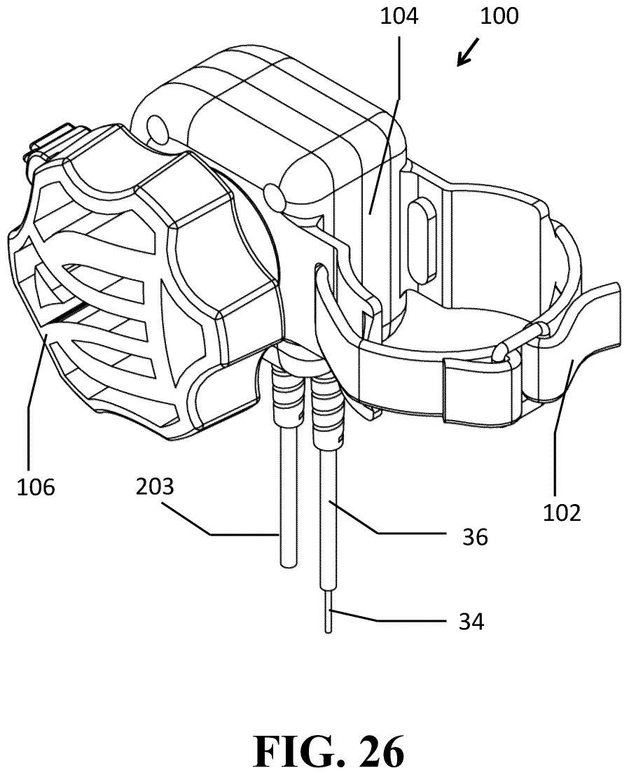

Referring now to FIGS. 25 and 26, embedded electronics of the user interface 100 are shown in greater detail, according to some embodiments of the present disclosure. In some embodiments, and as shown with particular reference to FIG. 2, the cable 203 is routed separately to the control unit 200 without being routed through the user interface 100. In other embodiments, the cable 203 may pass through the user interface 100 before being routed to the control unit 200. In any case, the cable 203 may interface with the control unit 200 via the connector 204, as shown with reference to FIGS. 2 and 17.

In some embodiments, the user interface 100 includes a processor 126 that may be used to condition electrical signals passed from the image sensor 20 (discussed above with reference to FIGS. 5 and 6) prior to transmitting such signals to the control unit 200. In some embodiments, such as those in which the image sensor 20 uses a serial protocol (e.g., i2c bus) to transmit data, the processor 126 includes circuity for extending the communication distance such that a long (e.g., two meter) cable may be used to connect the user interface 100 to the control unit 200. In other embodiments where the image sensor 20 uses analog voltage to transmit data, the processor 126 includes circuitry for filtering the analog signal prior to passing it to the off-board control unit 200. In some embodiments, the user interface 100 includes an oscillator circuit which generates a clock signal that may be used to drive a phase-locked loop (PLL) circuit embedded in the image sensor 20.

In further embodiments, the user interface 100 includes an LED heatsink 128, which may be an integrated heat-sink and LED combination configured to provide illumination to the endcap 10 via fiber optic coupling. The processor 126 may also include local voltage regulation for boosting or bucking the DC voltage provided by the control unit 200 to a level that is needed to drive the LED heatsink 128 and/or the image sensor 20. The processor 126 may also feature on-board memory (i.e. electrically erase-able programmable read-only memory, or EEPROM) that may be utilized to enforce single-based or subscription-based usage through electronic lockout. Such usage may be implemented, for example, by storing a unique device identifier and time stamp that is interrogated by the control unit 200 when the apparatus 400 is first activated. The processor may also feature on-board video decoding.

In some embodiments, the user interface 100 includes buttons that may be programmed to execute certain functions associated with video processing and acquisition. For example, a button on the user interface 100 may be configured to record a snapshot of the current view of the image sensor 20 for saving and storage in external media. Such buttons may additionally be configured to provide white balance, illumination brightness adjustment, image resolution adjustment, and auto-scaling in regards to the operation of the image sensor 20.

Referring again to FIGS. 2 and 3, the apparatus 400 is further discussed, according to some embodiments of the present disclosure. As mentioned above, the apparatus 400 may include the endcap 10, the user interface 100, and the control unit 200. The video processing unit may 200 may include a wired connection to the endcap 10, on-board power regulation (to convert the AC voltage supplied by the electrical grid to a suitable DC voltage), video processing and digital conversion circuitry (with optional software-based image interpolation to produce higher resolution than the native resolution of the CMOS sensor), video outputs to external monitors and/or frame grabbers in any number of different formats (HDMI, VGA, DVI, DisplayPort, S-Video, etc.), and various buttons/control inputs to allow a user to calibrate the image sensor 20, control the video's appearance (as captured by the image sensor 20), and modify video and image capture parameters of the image sensor 20 (including but not limited to device power, illumination brightness, white balancing, resolution, auto-gain/exposure, image capture and recording).

In such embodiments where the light source 22 is provided by an on-board LED housed within the endcap 10, the control unit 200 may be configured to provide adequate electrical power to the light source 22. In such embodiments where the light source 22 receives illumination from a component external to the endcap 10, the control unit 200 may include an illumination LED or incandescent bulb that is optically coupled to the light source 22 via an optical fiber bundle.

In some embodiments, the control unit 200 includes on-board storage for saving video recordings and image captures and snapshots as recorded by the user via the image sensor 20, as well as a means of extracting stored files (either by writing to an external storage medium, such as an external hard disk or SD card, or through serial transfer via universal serial bus (USB) connection). Alternatively the control unit may act as a pass-through to an external video/image capture and storage system, such as a frame grabbing system. The system may also have indicators that communicate device status and fault states, both visually (i.e. status LEDs and pilot lights) and audibly (i.e. alarm tones). The unit may also contain an RFID reader/antenna or requisite circuitry to interrogate any devices that get connected to the processor to determine compatibility and enforce lockout if compatibility is not confirmed (i.e. if the attached device is expired or is otherwise incompatible).

The endcap 10 may be a detachable endcap (e.g., configured to be removably attached to the distal end 302 of the endoscope 300), which features an integrated visualization and illumination sub-assembly 18 (configured to provide digital imaging and illumination), as well as a tool elevation mechanism. For example, the endcap 10 may include a collar portion 12, a leading portion 14, an elevator 16 (e.g., a lever, an arm, etc.)

In some embodiments, the user interface 100 attaches to the handle 301 of the endoscope 300, giving the physician control over the tool elevation mechanism of the endcap 10. The control unit 200 may converts the digital image sensor data into a format that can be displayed onto an external video monitor. Such a system effectively ‘transforms’ a standard forward-viewing endoscope into a duodenoscope for treating disorders of the biliary tract, by replicating the functions that are unique to the more specialized duodenoscope (specifically, tool elevation and side-viewing visualization) which are both infection-prone and expensive.

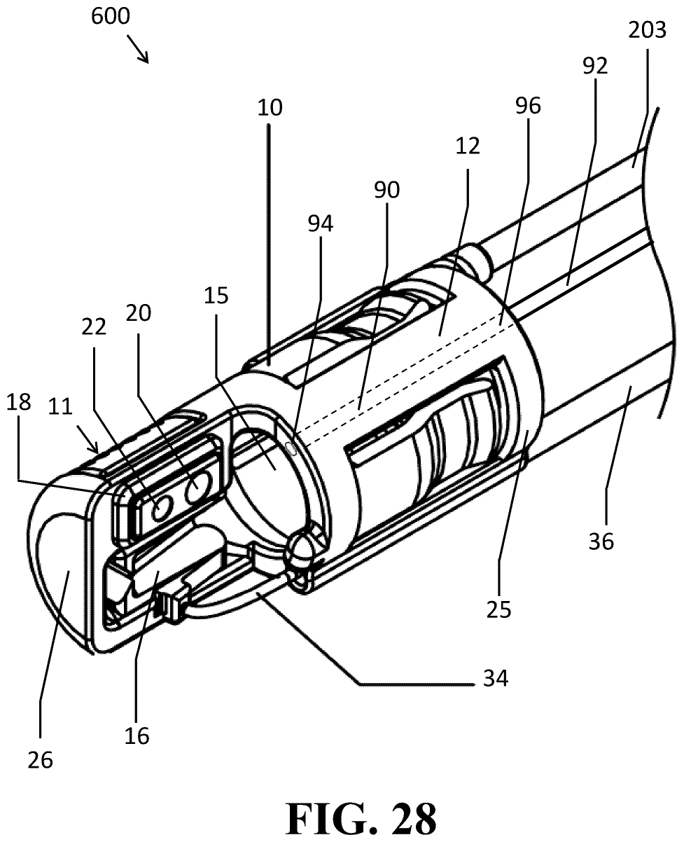

Referring now to FIG. 28, the endcap 10 is shown with a feature to clean the visualization and illumination assembly 18, according to some embodiments of the present disclosure. As mentioned above, the endcap 10 may include the visualization and illumination assembly 18, which may include the image sensor 20. In some embodiments, the endcap 10 further includes a feature for cleaning the visualization and illumination assembly 18 or, more particularly, the image sensor 20. For example, and as suggested above with reference to FIGS. 5-6, the endcap 10 may include an irrigation channel 90 that is configured to clean off the image sensor 20 when such the irrigation channel 90 is in communication with an external irrigation source at the proximal end of the device (e.g., at the proximal end of the endoscope 300). In some embodiments, the irrigation channel 90 is formed on the body 11 of the endcap 10, and is configured to provide a jet or stream of water for cleaning the image sensor 20. In turn, the endoscope 300 may include a long polymer tube 92 arranged to carry water to the irrigation channel 90 formed on the endcap 10. Alternatively, the long polymer tube 92 may be defined as a channel formed in the body of the endoscope 300 itself, such as the working port 304. Further, the long polymer tube 92 may simply be the working port 304.

The long polymer tube 92 may be configured to carry water from an off-board irrigation source to the endcap 10. A distal outlet 94 of the irrigation channel 90 is designed to spray a stream of water across the image sensor 20 to knock off any fluid, debris or fouling to restore a clear view. A proximal end 96 of the irrigation channel 90 (or a proximal end of the long polymer tube 92) may feature a standard Luer fitting which may be attached to an off-board irrigation system, including but not limited to a syringe, single-action irrigation pump, an automated irrigation system, or a gravity bag with a pressure cuff.

In further embodiments, the endoscope 300 is configured to clean the visualization and illumination assembly 18. In this sense, another method to clean the illumination and visualization assembly 18 is to leverage an auxiliary water jet feature that may already present in many front-facing endoscopes. This feature is commonly configured to spray a stream or jet of water axially out of the tip of the endoscope (e.g., the distal tip 303 of the endoscope 300, via the working port 304 for example) and is used to deliver irrigant to the surgical field for cleaning. However, the endcap 10 may be positioned in a way to leverage the auxiliary water jet's function to clean the image sensor 20 of the illumination and visualization assembly 18. The endcap 10 may be positioned and oriented in a way such that the elevator 16 obstructs the auxiliary water jet stream when deflected, resulting in the stream to be re-directed backwards and toward the illumination and visualization assembly 18, thereby serving the function of self-cleaning.

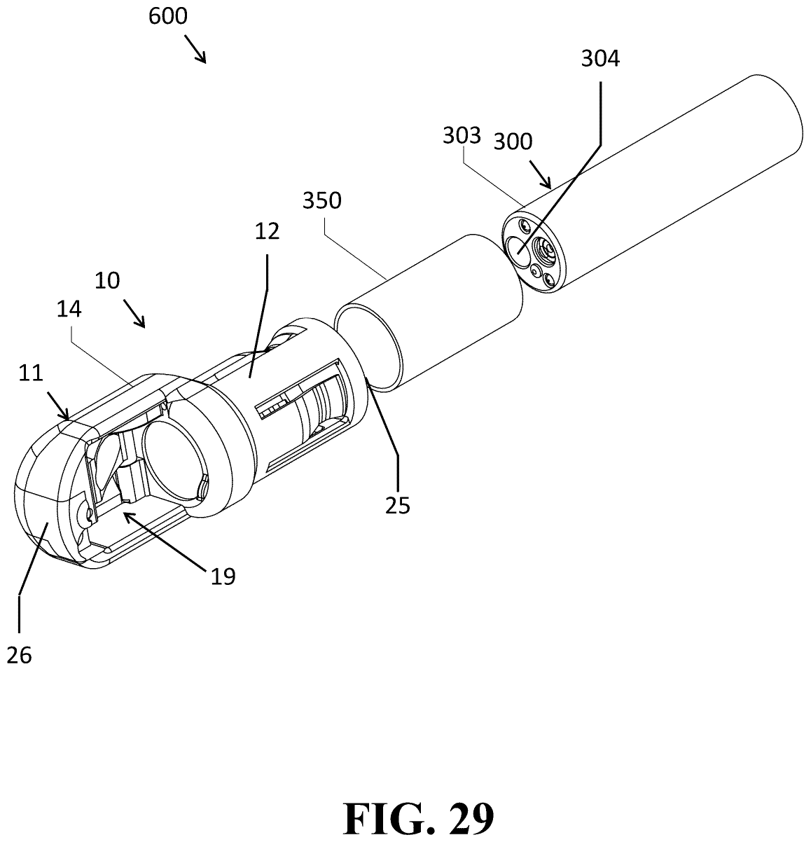

Referring now to FIG. 29, the apparatus 600 is shown, according to further embodiments of the present disclosure. As mentioned above with reference to FIG. 4, the endcap 10 may be removably secured to the distal end 302 of the endoscope 300. For instance, as discussed above with reference to FIGS. 5-6, the collar portion 12 (and thus the endcap 10 in its entirety) may be mechanically attached to the distal end 302 of the endoscope 300 through a removable mechanical fixation method that is non-damaging to the endoscope 300 and the endcap 10. In further embodiments, the apparatus 600 includes an intermediate sleeve 350, which may be arranged to facilitate securing the endcap 10 to the distal end 302 of the endoscope 300.

In some embodiments, in order to secure the endcap 10 to the distal end 302 of the endoscope 300, the intermediate sleeve 350 is first applied to the endoscope 300 (e.g., on the distal end 302 thereof), and the endcap 10 is then press-fit over the intermediate sleeve 350 to affix the endcap 10 to the endoscope 300 via the intermediate sleeve 350. In this sense, the apparatus 600 may include the intermediate sleeve 350 disposed between the endcap 10 and the endoscope 300.

In some embodiments, the intermediate sleeve 350 is constructed from a suitably soft, low-durometer material, such as silicone rubber or thermoplastic elastomer. The intermediate sleeve 350 may be designed to provide an interference fit with the outer surface of the endoscope 300, and an interference fit with the inner surface of the endcap 10 (e.g., the inner surface of the collar portion 12 of the endcap 10). In other words, the intermediate sleeve 350 may be disposed between the endcap 10 and the endoscope 300 such that an interference fit is formed between the endcap 10 and the endoscope 300.

Depending on the implementation, the amount of engagement (e.g., the coefficient of friction effectuated by the aforementioned interference fit) may be sufficient to ensure that the endcap 10 is suitably affixed to the endoscope 300 via the intermediate sleeve 350. In various embodiments, the endcap 10 (or, more particularly, the collar portion 12 of the endcap 10) feature ribs, ridges, or textures on its internal surface to engage with the intermediate sleeve 350 and increase the friction to ensure that the endcap 10 is stably affixed thereto.

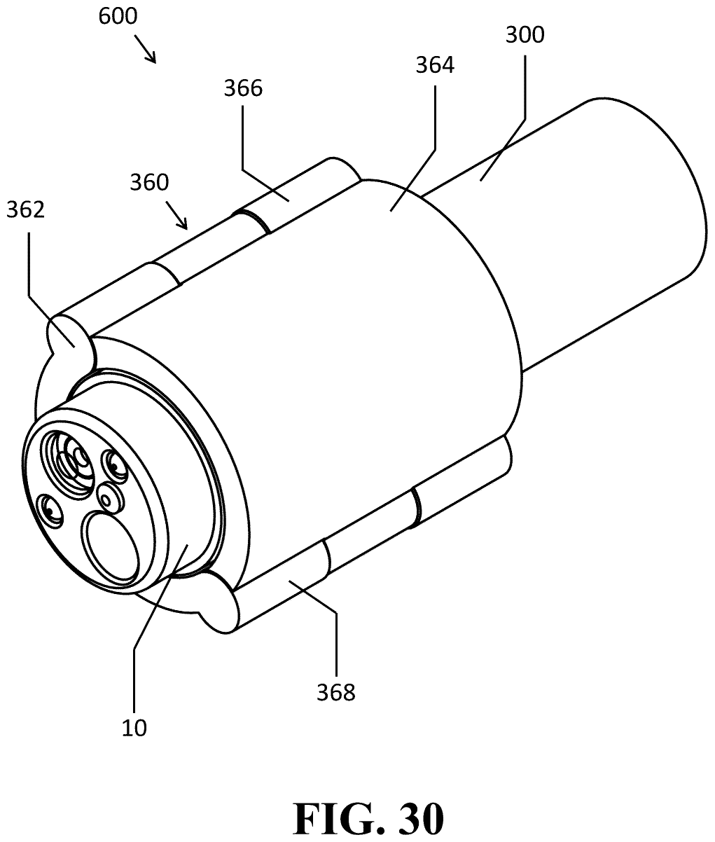

Referring now to FIGS. 30-32, the apparatus 600 is shown, according to further embodiments of the present disclosure. As suggested above, the apparatus 600 may include features arranged to assist or facilitate removably securing the endcap 10 to the endoscope 300. In further embodiments, the apparatus 600 further includes a clasp 360 that is configured to clamp the endcap 10 to the endoscope 300. In other words, the apparatus 600 may include the clasp 360 disposed about the endcap 10, and the clasp 360 may be configured to press the endcap 10 into the surface of the endoscope 300. Depending on the implementation, the clasp 360 may be a component of the endcap 10. In some embodiments, the clasp 360 includes a first clasp component 362 coupled to a second clasp component 364 via a hinge 366 on a closed end of the clasp 360. The first clasp component 362 can be secured to the second clasp component 364 on the open end of the clasp 360 via a latch 368 (e.g., an integrated closure and/or locking mechanism). Accordingly, the clasp 360 may be a hinged mechanism, such that the clasp 360 may be closed around the endoscope 10 and locked around the endoscope 10 via the latch 368.

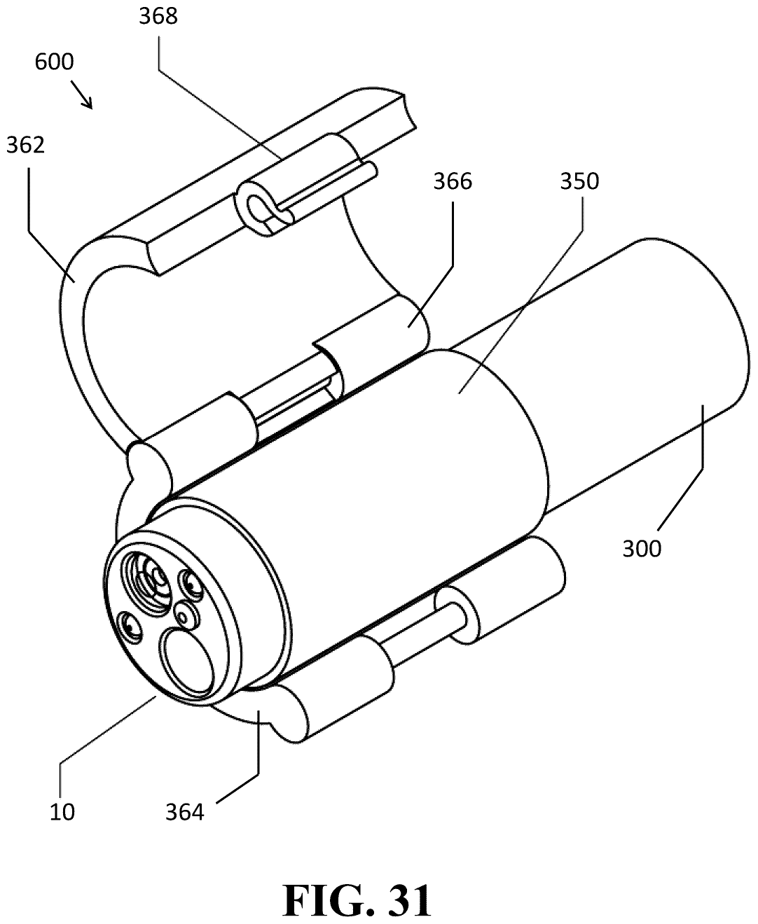

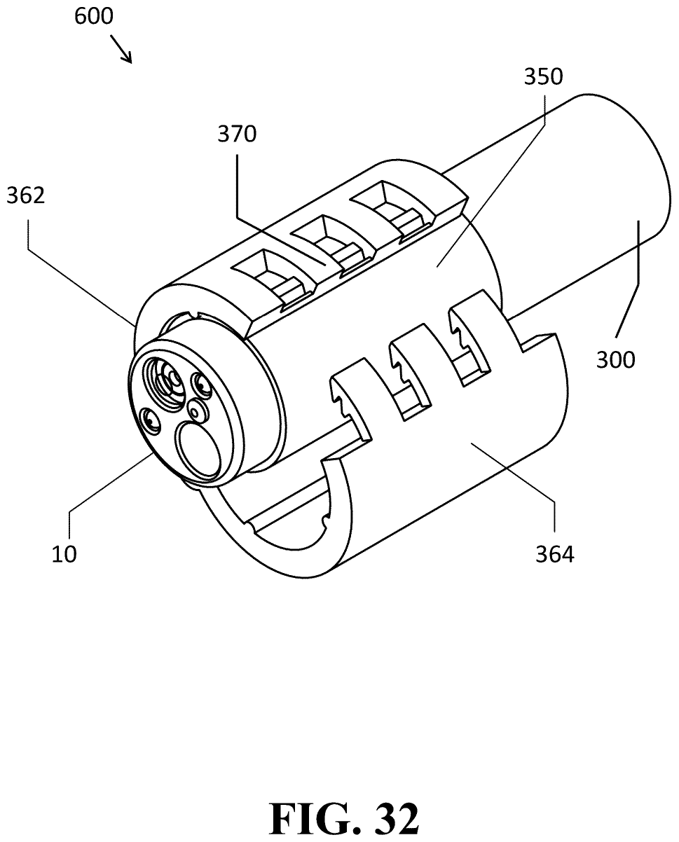

In some embodiments, and as shown with particular reference to FIGS. 31 and 32, the clasp 360 may be closed around the intermediate sleeve 350 which, as discussed above, may be positioned about the endoscope 10 and the endoscope 300. In other words, the apparatus 600 may include the clasp 360 disposed about the endcap 10, and the clasp 360 may be configured to press the endcap 10 into the surface of the intermediate sleeve 350, such that the intermediate sleeve 350 is pressed into the surface of the endoscope 300.

In other embodiments, the clasp 360 is a living hinge mechanism, and thus features a ratcheting mechanism 370 which enables the user to successively tighten the intermediate sleeve 350 and/or the endcap 10 around the endoscope 300. Depending on the implementation, by utilizing a living hinge (e.g., the clasp 360 with the ratcheting mechanism 370), the endcap 10 may be molded as a single monolithic piece.

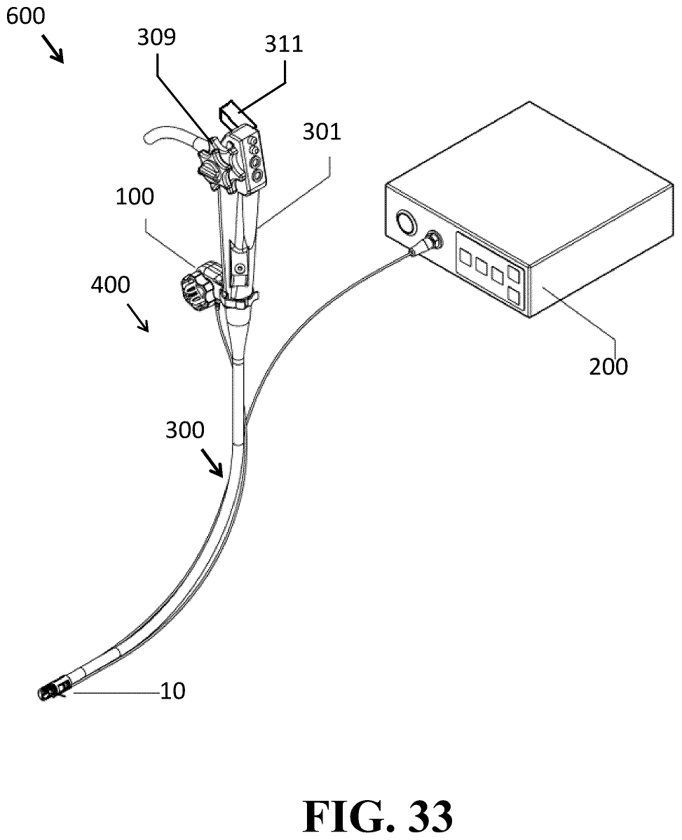

Referring now to FIG. 33, the handle 301 may include a lever 311, according to some embodiments of the present disclosure. For instance, the handle 301 may configured such that the user does not require their free hand to actuate the elevator 16. In a typical endoscopy workflow, the physician uses one hand (e.g., a “control hand”) to bear the weight of the endoscope 300 and operate the controls of the endoscope 300 (e.g., a control wheel 309 of the endoscope 300), and the other hand (e.g., a “free hand”) to guide the insertion of the endoscope 300 into the patient, or feed various tools into the working channel of the endoscope 300 (e.g., towards the working port 304 of the endoscope 300). Duodenoscope handles typically feature an extra lever near the control wheel 309 that, as applied to the present disclosure, could be actuated by the physician's thumb on their control hand to operate the elevator 16.

In some embodiments, the lever 311 is positioned in close proximity to the endoscope control wheel(s) 309, such that the user may use their control hand to also deflect the elevator 16. Such an embodiment may function similarly to a brake lever on a bicycle. In this sense, depressing the lever 311 applies a tensile force to the pull-wire 34 coupled to the elevator 16, thereby deflecting the elevator 16 in the endcap 10. In further embodiments, this feature may be implemented electronically, wherein the lever 311 is replaced with a button or a potientiometer, which sends a control signal to a motor contained within the rest of the handle 301 which is rotatably coupled to the pull-wire 34 coupled to the elevator 16. In further embodiments, the lever 311 is disposed on the user interface 100.

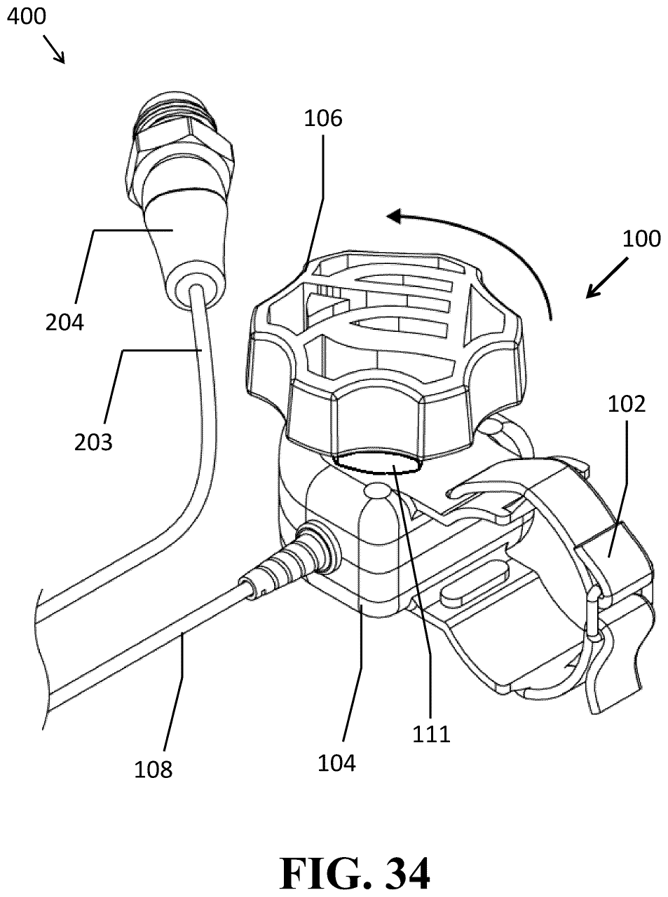

Referring now to FIG. 34, the user interface 100 may include a signal mechanism 111 that signals to the user when the elevator 16 has adequately deflected to lock the guidewire 306a, according to some embodiments of the present disclosure. Depending on the implementation, the signal mechanism 111 may be a tactile or audible element. For example, the signal mechanism 111 may be a detent or ratcheting feature that engages with a raised feature on the elevator deflection knob or lever (e.g., the knob 106), such that when the knob 106 is rotated by a certain amount (e.g., the amount required to lock the elevator 16), the energy stored in the signal mechanism 111 is suddenly released, resulting in an audible ‘click’ noise and/or a tactile sensation that may be felt by the user manipulating the knob 106 or lever.

In accordance with the discussion above regarding the apparatus 600, the apparatus 400, and the endcap 10, the present disclosure further provides for a method of performing surgery. The method may include providing the endcap 10 having the body 11 and the side opening 19. The method may include securing the body 11 of the endcap 10 onto the distal end 32 of the endoscope 300. The endoscope 300 may include the working port 304. The method may further include advancing the endoscopic tool 306 out of the working port 304. The method may include visualizing, via the image sensor 20 disposed on the body 11 of the endcap 10, the workspace that is lateral of both the endcap 10 and the distal end 302 of the endoscope 300 (e.g., the lateral workspace 514). The method may include pivoting the elevator 16 coupled to the body 11 of the endcap 10, such that the elevator 16 deflects the tool 306 towards the side opening 19 of the endcap 10 and into the lateral workspace 514.

In some embodiments, the method includes securing the user interface 100 to the endoscope 300 and providing the pull-wire 34 coupled to the user interface 100 and the elevator 16. The method may further include displacing the pull-wire 34 via the user interface 100, such that the elevator 16 is pivoted relative to the body 11 of the endcap 10 in response to displacement of the pull-wire 34. The elevator 16 may be configured to be pivoted relative to the body 11 of the endcap 10 in response to displacement of the pull-wire 34.

In the drawings, not all reference numbers are included in each drawing, for the sake of clarity. In addition, positional terms such as “upper,” “lower,” “side,” “top,” “bottom,” etc. refer to the apparatus when in the orientation shown in the drawing, or as otherwise described. A person of skill in the art will recognize that the apparatus can assume different orientations when in use.

Thus, although there have been described particular embodiments of the present invention of a new and useful SIDE-VIEWING ENDOSCOPE CAP, it is not intended that such references to particular embodiments be construed as limitations upon the scope of this invention.

What is claimed is:

1. An apparatus for an endoscope, comprising:
an endcap including a body and a side opening, the body configured to fit onto a distal end of an endoscope, wherein the endoscope includes a forward-facing working port configured for advancement of a tool from the working port;
an image sensor disposed on the body of the endcap, the image sensor configured to visualize a workspace; and
an elevator pivotably coupled to the body of the endcap, the elevator configured to be pivoted relative to the body such that the elevator deflects the tool toward the side opening of the endcap into the workspace.

2. The apparatus of claim 1, wherein the image sensor is positioned on the body of the endcap such that the image sensor visualizes the workspace through the side opening of the endcap.

3. The apparatus of claim 1, wherein the elevator is configured to deflect the tool until the tool is secured in place between the elevator and an edge of the side opening.

4. The apparatus of claim 3, wherein when the tool is secured in place between the elevator and the edge of the side opening, the tool resists advancement from or retraction into the working channel.

5. The apparatus of claim 1, further comprising:
a user interface configured to be secured to the endoscope; and
a pull-wire coupled to the user interface and the elevator,
wherein the user interface is configured to displace the pull-wire, and
wherein the elevator is configured to be pivoted relative to the body of the endcap in response to displacement of the pull-wire.

6. The apparatus of claim 5, wherein the elevator is configured to be pivoted towards the working port when the pull-wire is displaced towards the user interface, and
wherein the elevator is configured to be pivoted away from the working port when the pull-wire is displaced away from the user interface.

7. The apparatus of claim 1, wherein the image sensor includes a complementary metal-oxide-semiconductor sensing array.

8. The apparatus of claim 1, further comprising a light source disposed on the body of the endcap and configured to illuminate the workspace.

9. A method of performing surgery, the method comprising:
providing an endcap including a body and a side opening,
securing the body onto a distal end of an endoscope, wherein the endoscope includes a forward-facing working port;
advancing a tool out of the working port;
visualizing, via an image sensor disposed on the body of the endcap, a workspace that is lateral of both the endcap and the distal end of the endoscope;
pivoting an elevator coupled to the body of the endcap, such that the elevator deflects the tool towards the side opening of the endcap into the workspace.

10. The method of claim 9, wherein the image sensor is positioned on an interior of the body of the endcap, such that the image sensor visualizes the workspace through the side opening of the endcap.

11. The method of claim 9, wherein the elevator is configured to deflect the tool toward the side opening of the endcap such that the tool is secured in place between the elevator and an edge of the side opening.

12. The method of claim 11, wherein when the tool is secured in place between the elevator and the edge of the side opening, the tool resists advancement from or retraction into the working channel.

13. The method of claim 9, further comprising:
securing a user interface to the endoscope;
providing a pull-wire coupled to the user interface and the elevator; and
displacing the pull-wire via the user interface, such that the elevator is pivoted relative to the body of the endcap in response to displacement of the pull-wire,
wherein the elevator is configured to be pivoted relative to the body of the endcap in response to displacement of the pull-wire.

14. The method of claim 13, wherein the elevator is configured to be pivoted towards the working port when the pull-wire is displaced towards the user interface, and
wherein the elevator is configured to be pivoted away from the working port when the pull-wire is displaced away from the user interface.

15. The method of claim 9, wherein the image sensor includes a complementary metal-oxide-semiconductor sensing array.

16. The method of claim 9, further comprising illuminating the workspace via a light source disposed on the body of the endcap.

17. An apparatus for an endoscope, comprising:
an endcap including a body and a side opening, the body configured to fit onto a distal end of an endoscope, wherein the endoscope includes a forward-facing working port configured to advance a tool into a workspace;
an elevator pivotably coupled to the body of the endcap, the elevator configured to be pivoted relative to the body such that the elevator deflects the tool toward the side opening of the endcap into the workspace;
a user interface configured to be secured to the endoscope; and
a pull-wire coupled to the user interface and the elevator,
wherein the user interface is configured to displace the pull-wire, and
wherein the elevator is configured to be pivoted relative to the body of the endcap in response to displacement of the pull-wire.

18. The apparatus of claim 17, further comprising an image sensor disposed on the body of the endcap, the image sensor configured to visualize a portion of the workspace that is lateral of both the endcap and the distal end of the endoscope.

19. The apparatus of claim 18, wherein the image sensor includes a complementary metal-oxide-semiconductor sensing array.

20. The apparatus of claim 18, further comprising a light source disposed on the body of the endcap and configured to illuminate the workspace.
